SEQUENCE LISTING (75 sequences)

SEQ ID NO: 1 - protein, 99 aa, Homo sapiens [sequence omitted]

SEQ ID NO: 2 - protein, 99 aa, synthetic construct
IQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 3 - protein, 275 aa, Homo sapiens [sequence omitted]

SEQ ID NO: 4 - protein, 275 aa, synthetic construct
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 5 - protein, 275 aa, synthetic construct
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 6 - protein, 275 aa, synthetic construct
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 7 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 12 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 13 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 14 - protein, 133 aa, Homo sapiens [sequence omitted]

SEQ ID NO: 15 - protein, 158 aa, synthetic construct
MHQKRTAMFQDPQERPRKLPQLCTELQTTIHDIILECVYCKQQLLRREVYDFAFRDLCIVYRDGNPYAVCDKCLKFYSKISEYRHYCYSVYGTTLEQQYNKPLCDLLIRCINCQKPLCPEEKQRHLDKKQRFHNIRGRWTGRCMSCCRSSRTRRETQL

SEQ ID NO: 16 - protein, 141 aa, synthetic construct
MHGDTPTLHEYMLDLQPETTDLYCYEQLNYSSEEEDEIDGPAGQAEPDRAHYNIVTFCCKCDSTLRLCVQSTHVDIRTLEDLLMGTLGIVCPICSQKPYSTMADPAGTNGEEGTGCNGWFYVEAVVEKKTGDAISDDENEN

SEQ ID NO: 17 - protein, 813 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 812 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 124 aa, synthetic construct
YMLDLQPETTGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 20 - protein, 10 aa, synthetic construct
YMLDLQPETT

SEQ ID NO: 21 - protein, 133 aa, synthetic construct
  VARIANT 16: note = wherein X is an amino acid other than His
  VARIANT 42: note = wherein X is an amino acid other than Phe
APTSSSTKKTQLQLEXLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 22 - protein, 133 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 23 - protein, 133 aa, synthetic construct
APTSSSTKKTQLQLETLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 24 - protein, 9 aa, synthetic construct
KLPQLCTEL

SEQ ID NO: 25 - protein, 9 aa, synthetic construct
LQTTIHDII

SEQ ID NO: 26 - protein, 9 aa, synthetic construct
VYDFAFRDL

SEQ ID NO: 27 - protein, 9 aa, synthetic construct
FAFRDLCIV

SEQ ID NO: 28 - protein, 9 aa, synthetic construct
KFYSKISEY

SEQ ID NO: 29 - protein, 9 aa, synthetic construct
ISEYRHYCY

SEQ ID NO: 30 - protein, 9 aa, synthetic construct
TLHEYMLDL

SEQ ID NO: 31 - protein, 9 aa, synthetic construct
YMLDLQPET

SEQ ID NO: 32 - protein, 9 aa, synthetic construct
QAEPDRAHY

SEQ ID NO: 33 - protein, 9 aa, synthetic construct
RAHYNIVTF

SEQ ID NO: 34 - protein, 9 aa, synthetic construct
CDSTLRLCV

SEQ ID NO: 35 - protein, 10 aa, synthetic construct
LCVQSTHVDI

SEQ ID NO: 36 - protein, 9 aa, synthetic construct
LLMGTLGIV

SEQ ID NO: 37 - protein, 8 aa, synthetic construct
TLGIVCPI

SEQ ID NO: 38 - protein, 12 aa, synthetic construct
LLMGTLGIVCPI

SEQ ID NO: 39 - protein, 9 aa, synthetic construct
VYDFAFRDL

SEQ ID NO: 40 - protein, 9 aa, synthetic construct
CYSLYGTTL

SEQ ID NO: 41 - protein, 9 aa, synthetic construct
EYRHYCYSL

SEQ ID NO: 42 - protein, 9 aa, synthetic construct
KLPQLCTEL

SEQ ID NO: 43 - protein, 9 aa, synthetic construct
DPQERPRKL

SEQ ID NO: 44 - protein, 9 aa, synthetic construct
HYCYSLYGT

SEQ ID NO: 45 - protein, 9 aa, synthetic construct
DFAFRDLCI

SEQ ID NO: 46 - protein, 10 aa, synthetic construct
LYGTTLEQQY

SEQ ID NO: 47 - protein, 10 aa, synthetic construct
HYCYSLYGTT

SEQ ID NO: 48 - protein, 10 aa, synthetic construct
EVYDFAFRDL

SEQ ID NO: 49 - protein, 10 aa, synthetic construct
EYRHYCYSLY

SEQ ID NO: 50 - protein, 10 aa, synthetic construct
VYDFAFRDLC

SEQ ID NO: 51 - protein, 10 aa, synthetic construct
YCYSIYGTTL

SEQ ID NO: 52 - protein, 9 aa, synthetic construct
VYCKTVLEL

SEQ ID NO: 53 - protein, 9 aa, synthetic construct
VYGDTLEKL

SEQ ID NO: 54 - protein, 10 aa, synthetic construct
LTNTGLYNLL

SEQ ID NO: 55 - protein, 9 aa, synthetic construct
DLQPETTDL

SEQ ID NO: 56 - protein, 9 aa, synthetic construct
TLHEYMLDL

SEQ ID NO: 57 - protein, 9 aa, synthetic construct
TPTLHEYML

SEQ ID NO: 58 - protein, 9 aa, synthetic construct
RAHYNIVTF

SEQ ID NO: 59 - protein, 9 aa, synthetic construct
GTLGIVCPI

SEQ ID NO: 60 - protein, 9 aa, synthetic construct
EPDRAHYNI

SEQ ID NO: 61 - protein, 9 aa, synthetic construct
QLFLNTLSF

SEQ ID NO: 62 - protein, 9 aa, synthetic construct
FQQLFLNTL

SEQ ID NO: 63 - protein, 10 aa, synthetic construct
AFQQLFLNTL

SEQ ID NO: 64 - protein, 9 aa, synthetic construct
VYDFAFRDL

SEQ ID NO: 65 - protein, 9 aa, synthetic construct
RAHYNIVTF

SEQ ID NO: 66 - protein, 9 aa, synthetic construct
CDSTLRLCV

SEQ ID NO: 67 - protein, 10 aa, synthetic construct
LCVQSTHVDI

SEQ ID NO: 68 - protein, 55 aa, synthetic construct
  VARIANT 11..55: note = wherein up to 9 copies of GGGGS may be deleted
CGGGSGGGGSGGGGSGGGGSGGGGSGGGGSGGGGSGGGGSGGGGSGGGGSGGGGS

SEQ ID NO: 69 - protein, 55 aa, synthetic construct
  VARIANT 11..55: note = wherein up to 9 copies of GGGGS may be deleted
GCGGSGGGGSGGGGSGGGGSGGGGSGGGGSGGGGSGGGGSGGGGSGGGGSGGGGS

SEQ ID NO: 70 - protein, 55 aa, synthetic construct
  VARIANT 11..55: note = wherein up to 9 copies of GGGGS may be deleted
GGCGSGGGGSGGGGSGGGGSGGGGSGGGGSGGGGSGGGGSGGGGSGGGGSGGGGS

SEQ ID NO: 71 - protein, 15 aa, synthetic construct
GGGGSGGGGSGGGGS

SEQ ID NO: 72 - protein, 50 aa, synthetic construct
  VARIANT 6..50: note = wherein up to 9 copies of GGGGS may be deleted
GGGGSGGGGSGGGGSGGGGSGGGGSGGGGSGGGGSGGGGSGGGGSGGGGS

SEQ ID NO: 73 - protein, 5 aa, synthetic construct
AAAGG

SEQ ID NO: 74 - protein, 20 aa, synthetic construct
GGGGSGGGGSGGGGSGGGGS

SEQ ID NO: 75 - protein, 15 aa, synthetic construct
GGGGSGGGGSGGGGS

(19) United States
(12) Patent Application Publication
(10) Pub. No.: US 2026/0240979 A1
(43) Pub. Date: Aug. 20, 2026

(54) Methods for Treating HPV16-Associated Cancers

(71) Applicant: Cue Biopharma, Inc., Boston, MA (US)

(72) Inventors: Matteo G. Levisetti, Boston, MA (US); Steven Paul Margossian, Boston, MA (US)

(21) Appl. No.: 19/664,315

(22) Filed: Apr. 30, 2026

Related U.S. Application Data
(63) Continuation of application No. PCT/US2024/054578, filed on Nov. 5, 2024.
(60) Provisional application No. 63/547,509, filed on Nov. 6, 2023.

(51) Int. Cl. A61K 39/12; A61K 39/00; A61K 39/385; A61K 39/395; A61P 37/04

(52) U.S. Cl. CPC A61K 39/12; A61K 39/385; A61K 39/3955; A61P 37/04; A61K 2039/545; A61K 2039/55516; A61K 2039/55533; A61K 2039/585; A61K 2039/605

(57) ABSTRACT

This disclosure provides a method of treating an HPV16-associated cancer in an individual, the method comprising administering to the individual a T-cell modulatory protein (TMP) and an PD-(L)1 CPI that targets PD-1 or PD-L1, i.e., a “PD-(L)1 CPI”, where (A) the individual previously was determined to have progressive disease based on RECIST 1.1 criteria following (i) monotherapy with a PD-(L)1 CPI, or (ii) combination therapy comprising treatment with a PD-(L)1 CPI and a therapeutic agent other than the TMP, or (B) where the individual has a PD-L1 Combined Positive Score (PD-L1 CPS) of 0 or more than 0 but less than 1.

### CROSS-REFERENCE TO RELATED APPLICATIONS

This application is a continuation of PCT Application No. PCT/US2024/054578, filed Nov. 5, 2024, which claims the benefit of U.S. Provisional Patent Application No. 63/547,509, filed Nov. 6, 2023, which applications are incorporated herein by reference in their entirety.

### INCORPORATION BY REFERENCE OF SEQUENCE LISTING

A Sequence Listing is provided herewith as a Sequence Listing XML, “CUEB-160WO_SEQLIST.xml” created on Nov. 4, 2024 and having a size of 74,080 bytes. The contents of the Sequence Listing XML are incorporated herein by reference in their entirety.

### INTRODUCTION

Cytotoxic T cells (CTLs) are activated T cells that play an important role in the immune system's defense against cancer. When a CTL recognizes and binds to a target antigen expressed on the surface of a cancer cell, it recognized the cancer cell as a foreign cell and releases its payload of cytotoxins, which induce apoptosis (cell death) in the target cancer cell.

Immune checkpoints are a type of protein on the surface of some immune cells such as CTLs that act as a switch to regulate the immune response. Immune checkpoints are important for maintaining self-tolerance, which prevents the immune system from attacking normal cells in the body. They do this by binding to their inhibitory co-receptors found on healthy cells. The binding of a checkpoint to its inhibitory co-receptor on a healthy cell signals the CTL to deactivate and not destroy the healthy cell in an immune response (i.e., an autoimmune response).

One of the most important checkpoints is PD-1 (programmed death receptor-1), which is expressed on CTLs. When PD-1 binds to its inhibitory co-receptor PD-L1 (programmed death receptor ligand 1) on a healthy cell, it will cause the CTL to deactivate and not release its cytotoxins to kill the healthy cell. Unfortunately, however, some cancer cells also can express PD-L1, which allows them to evade the immune system and grow unchecked.

Immune checkpoint inhibitors (CPIs) that interfere with the binding of PD-1 to PD-L1 have thus become an important weapon in the fight against cancers that express PD-L1. Such CPIs, which are denoted “PD-(L)1 CPIs” commonly are antibodies that bind either to PD-1 on CTLs or to PD-L1 on cancer cells. By blocking the binding of PD-1 on a CTL to PD-L1 on a cancer cell, these antibodies prevent deactivation of CTLs, which allows the CTL to release its cytotoxins and kill the cancer cell. In this way, CPIs actually help a patient's own immune system to detect and eliminate cancer cells.

PD-(L)1 CPIs have been tested alone or in combination with other anti-cancer agents in a large number of clinical trials. Among other things, these trials have revealed that (i) monotherapy with PD-(L)1 CPIs is not effective for all cancers, and (ii) for cancers that do respond to anti-PD-1 monotherapy, the response can vary widely among patients, especially among patients whose cancers express either high or low amounts of PD-L1.

One such clinical trial was the KEYNOTE-048 trial for patients who have recurrent/metastatic head and neck squamous cell carcinoma (R/M HNSCC). Head and neck cancers can have multiple causes, including human papillomavirus (HPV) infections. HPV16 infections, in particular, are responsible for a high percentage of all head and neck cancers, as well as other types of HPV-associated cancers such as cervical cancer, anal cancer, vulvar cancer, and vaginal cancer.

In KEYNOTE-048, one cohort of patients received monotherapy with the anti-PD-1 antibody pembrolizumab (KEYTRUDA®). The overall response rate (ORR) of these patients, as measured by standard RECIST v1.1 criteria, varied widely for patients depending on their PD-L1 Combined Positive Score (PD-L1 CPS), which is a measure of PD-L1 expression on cancer cells. The ORR was significantly better for patients who had a higher PD-L1 CPS of 20 or greater (23.3%), than for patients who had a lower PD-L1 CPS of from 1 to 19 (14.5%), and far better than for patients whose CPS scores were below 1 or negative (4.5%). See, Harrington et al., J. Clin. Oncol. 41:790-802 (2022), and Burtness et al. J. Clin. Oncol. 40:2321-2332 (2022). Notably, it was found that that ~42% of patients in the trial had a CPS of 1-19, ~43% of the patients had a CPS≥20, and ~14.5% of patients had a CPS<1. As a result of the KEYNOTE-048 trial, pembrolizumab (KEYTRUDA®) is now an approved treatment as a single agent for the first-line treatment of patients with metastatic or with unresectable, recurrent HNSCC whose tumors express PD-L1 CPS≥1 as determined by an FDA-approved test.

Another clinical trial involving an anti-PD-1 monotherapy was the CHECKMATE-141 trial in which patients with recurrent or metastatic squamous cell carcinoma of the head and neck (R/M SCCHN) who experienced disease progression on or within 6 months after platinum-based therapy received nivolumab or the investigator's choice of chemotherapy. Patients who received nivolumab experienced significantly improved overall survival (OS) compared with the investigator's choice of chemotherapy. The ORR for the nivolumab patients was reported to be 20%. See, Gillison et al, The Oncologist 27 (2), e194-e198 (2022). Nivolumab (OPDIVO®) is now approved for the treatment of adult patients with recurrent or metastatic squamous cell carcinoma of the head and neck (SCCHN) with disease progression on or after platinum-based therapy.

Accordingly, there is a need for additional treatment options for the significant percentage of head and neck cancer patients with HPV16+R/M HSNCC who do not qualify for monotherapy with pembrolizumab or for whom anti-PD-1 monotherapy or anti-PD-1 combination therapy with one or more chemotherapeutics has been unsuccessful. There is also a need for additional treatment options for HPV16-associated cancers in general, including, e.g., cervical cancer, oropharyngeal cancers, anal cancer, vulvar and vaginal cancers.

### SUMMARY

The present disclosure provides methods of treating a patient who has an HPV16-associated cancer, the methods including administering to the individual a T-cell modulatory protein (TMP) and a PD-(L)1 CPI, (A) where the individual previously was determined to have progressive disease based on RECIST 1.1 criteria following (i) monotherapy with a PD-(L)1 CPI, or (ii) combination therapy comprising treatment with a PD-(L)1 CPI and one or more therapeutic agents (e.g., one or more chemotherapeutic agents) other than the TMP, or (B) in any line of treatment where the patient has received a PD-L1 Combined Positive Score (PD-L1 CPS) of 0 or more than 0 but less than 1. The present disclosure also provides methods of treating a patient who has any HPV16-associated cancer, including, e.g., cervical cancer, a head and neck cancer, anal cancer, and vulvar and vaginal cancers.

### BRIEF DESCRIPTION OF THE DRAWINGS

FIG. 1 illustrates the best change from baseline in the sum of diameters of target lesions with best overall response as measured by RECIST 1.1 for 17 evaluable patients who received a combination of a TMP (CUE-101) and a PD-(L)1 CPI (pembrolizumab) as described in Example 1.

FIG. 2 is a spider plot that shows change from baseline in the sum of diameters of target lesions as measured by RECIST 1.1 for 17 evaluable patients who received a combination of a TMP (CUE-101) and a PD-(L)1 CPI (pembrolizumab) as described in Example 1.

FIG. 3 is a swimmer plot illustrating the overall survival (months from first dose) in patients who received a combination of a TMP (CUE-101) and a checkpoint inhibitor (pembrolizumab) as described in Example 1.

FIG. 4 illustrates the data discussed in Example 1 for the ORR, as determined by RECIST 1.1 criteria, for patients who have PD-L1 Combined Positive Score (PD-L1 CPS)≥land who received a combination of a TMP (CUE-101) and a PD-(L)1 CPI (pembrolizumab) compared to the ORR reported in the KEYNOTE-048 clinical trial for patients who received pembrolizumab alone. See, Harrington et al., J. Clin. Oncol. 41:790-802 (2022), and Burtness et al. J. Clin. Oncol. 40:2321-2332 (2022).

FIG. 5 illustrates the data discussed in Example 1 for the ORR, as determined by RECIST 1.1 criteria, for patients who have a PD-L1 CPS from 1-19, as determined by RECIST 1.1 criteria, who received a combination of a TMP (CUE-101) and a PD-(L)1 CPI (pembrolizumab) compared to the ORR reported in the KEYNOTE-048 clinical trial for patients who received pembrolizumab alone.

FIG. 6 illustrates the data discussed in Example 1 for the ORR, as determined by RECIST 1.1 criteria, for patients who have a PD-L1 CPS≥20 who received a combination of a TMP (CUE-101) and a PD-(L)1 CPI (pembrolizumab) compared to the ORR reported in the KEYNOTE-048 clinical trial for patients who received pembrolizumab alone.

FIG. 7A-7V provide amino acid sequences of B2M polypeptides (FIGS. 7A and 7B); MHC class I heavy chain polypeptides (FIG. 7C-7F); immunoglobulin (Ig) Fc polypeptides (FIG. 7G-7J); IL-2 receptor polypeptides (FIG. 7K-7M); wild-type IL-2 polypeptide (FIG. 7N); a human papillomavirus (HPV) E6 polypeptide (FIG. 7O); an HPV E7 polypeptide (FIG. 7P); amino acid sequences of TMPs (FIG. 7Q-7S; linkers: (GGGGS)3, (GGGGS)4, and AAAGG are SEQ ID NOs: 71, 74, and 73, respectively); and amino acid sequences of variant IL-2 polypeptides (FIG. 7T-7V).

### DEFINITIONS

The term “Approved Laboratory” means a laboratory that has been certified under the Clinical Laboratory Improvement Acts (CLIA) or has been certified under a state licensure program in a state that is exempt under CLIA (currently the states of New York and Washington). Where an assay for PD-L1 CPS is performed outside of the U.S., an Approved Laboratory is a laboratory that is approved and/or certified under the laws of the jurisdiction where the laboratory is located.

The term “PD-L1 CPS” as used herein means a Combined Positive Score for PD-L1 expression received from any Approved Laboratory. An Approved Laboratory may or may not use an approved companion diagnostic assay to determine the PD-L1 CPS. For example, a PD-L1 CPS may be determined by an FDA-approved companion diagnostic, e.g., an approved PD-L1 IHC 22C3 pharmDx, which employs a monoclonal mouse anti-PD-L1 antibody, clone 22C3, or an approved PD-L1 IHC 28-8 pharmDx, which employs a monoclonal mouse anti-PD-L1 antibody, clone 28-8, or any other PD-L1 CPS assay that is performed by an Approved Laboratory. Alternatively, an Approved Laboratory may use a different assay and/or a different antibody to determine the PD-L1 CPS.

Amino acid sequences of the heavy and light chain variable regions of anti-PD-L1 antibody clone 22C3 are described in, e.g., U.S. Pat. No. 9,709,568.

Amino acid sequences of the heavy and light chain variable regions of anti-PD-L1 antibody clone 28-8 is described in, e.g., U.S. Pat. No. 9,212,224.

The term “CPI” means an antibody that acts as an immune checkpoint inhibitor by binding to an immune checkpoint protein (e.g., PD-1) and blocking it from binding with its co-receptor (e.g., PD-L1).

The term “PD-(L)1 CPI” means (i) a CPI that binds to PD-1 and blocks it from binding to PD-L1, or (ii) a CPI that binds to PD-L1 and blocks it from binding to PD-1.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. Furthermore, as used herein, a “polypeptide” refers to a protein that includes modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to polymerase chain reaction (PCR) amplification or other recombinant DNA methods. References herein to a specific residue or residue number in a known polypeptide are understood to refer to the amino acid at that position in the wild-type polypeptide. To the extent that the sequence of the wild-type polypeptide is altered, either by addition or deletion of one or more amino acids, one of ordinary skill will understand that a reference to the specific residue or residue number will be correspondingly altered so as to refer to the same specific amino acid in the altered polypeptide, which would be understood to reside at an altered position number. For example, if an MHC class I polypeptide is altered by the addition of one amino acid at the N-terminus, then a reference to position 84 or a specific residue at position 84, will be understood to indicate the amino acids that are at position 85 on the altered polypeptide. Likewise, a reference herein to substitution of a specific amino acid at a specific position, e.g., Y84, is understood to refer to a substitution of an amino acid for the amino acid at position 84 in the wild-type polypeptide. A Y84C substitution is thus understood to be a substitution of Cys residue for the Tyr residue that is present in the wild-type sequence. If, e.g., the wild-type polypeptide is altered to change the amino acid at position 84 from its wild-type amino acid to an alternate amino acid, then the substitution for the amino acid at position 84 will be understood to refer to the substitution for the alternate amino acid. If in such case the polypeptide is also altered by the addition or deletion of one or more amino acids, then the reference to the substitution will be understood to refer to the substitution for the alternate amino acid at the altered position number. A reference to a “non-naturally occurring Cys residue” in a polypeptide, e.g., an MHC class I polypeptide, means that the polypeptide comprises a Cys residue in a location where there is no Cys in the corresponding wild-type polypeptide. This can be accomplished through routine protein engineering in which a cysteine is substituted for the amino acid that occurs in the wild-type sequence.

A polypeptide has a certain percent “sequence identity” to another polypeptide, meaning that, when aligned, that percentage of amino acids are the same, and in the same relative position, when comparing the two sequences. Sequence identity can be determined in a number of different ways. To determine sequence identity, sequences can be aligned using various convenient methods and computer programs (e.g., BLAST (Basic Local Alignment Search Tool), T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/. See, e.g., Altschul et al. (1990), J. Mol. Biol. 215:403-10. Unless otherwise stated, “sequence identity” as referred to herein is determined by BLAST+2.10.0 using default parameters, which is available over the world wide web at sites including https://ncbiinsights.ncbi.nlm.nih.gov/2019/12/18/blast-2-10-0/.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

“T cell” includes all types of immune cells expressing CD3, including T-helper cells (CD4+ cells), cytotoxic T-cells (CD8+ cells), T-regulatory cells (Treg), and NK-T cells.

The term “immunomodulatory polypeptide” (also referred to herein as a “MOD”), as used herein, means a polypeptide that specifically binds a cognate costimulatory polypeptide on a T cell, thereby providing a signal which, in addition to the primary signal provided by, for instance, binding of a TCR/CD3 complex with a major histocompatibility complex (MHC) polypeptide loaded with peptide, mediates a T cell response, including, but not limited to, proliferation, activation, differentiation, and the like. As discussed herein, a MOD can include, but is not limited to wild-type or variants of wild-type polypeptides such as a cytokine (e.g., IL-2), CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor, and a ligand that specifically binds with B7-H3. A MOD of a TMP can bind a cognate costimulatory polypeptide (i.e., a “co-MOD”) that is present on a target T cell. A MOD can be an antibody or a binding fragment of an antibody.

As used herein the term “in vivo” refers to any process or procedure occurring inside of the body.

As used herein, “in vitro” refers to any process or procedure occurring outside of the body.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may or may not be predisposed to acquiring the disease or symptom but has not yet been diagnosed as having it; (b) inhibiting the disease or one or more symptoms associated with the disease, e.g., arresting its development; and/or (c) relieving the disease, i.e., causing regression of the disease. The therapeutic agent may be administered before, during and/or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired. Mammals include, e.g., humans, non-human primates, rodents (e.g., rats; mice), lagomorphs (e.g., rabbits), ungulates (e.g., cows, sheep, pigs, horses, goats, and the like), etc. Unless otherwise indicated, the terms “individual,” “subject,” “host,” and “patient,” refer to a human.

Unless indicated otherwise, the term “substantially” is intended to encompass both “wholly” and “largely but not wholly”. For example, an Ig Fc that “substantially does not induce ADCC” (i.e., antibody-dependent cell cytotoxicity) means an Ig Fc that induces no ADCC at all or that largely does not induce ADCC.

As used herein, the term “about” used in connection with an amount indicates that the amount can vary by 10% of the stated amount. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

As used herein and in the appended claims, the singular forms “a,” “an,” and “the” include plural referents unless the context clearly dictates otherwise. Thus, for example, reference to a “T-cell modulatory protein” includes a plurality of such proteins and reference to “the immunomodulatory polypeptide” includes reference to one or more immunomodulatory polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the embodiments of the disclosure and does not pose a limitation on the scope of the disclosure unless otherwise claimed.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that aspects and embodiments of the present disclosure described herein include “comprising,” “consisting,” and “consisting essentially of” aspects and embodiments.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure prior to the filing date of the present application. Nothing herein is to be construed as an admission that the present disclosure is not entitled to antedate such publication. Further, the dates of publication provided may be different from the actual publication dates, which may need to be independently confirmed.

### DETAILED DESCRIPTION

The present disclosure provides methods of treating a patient who has HPV16-associated cancer, the methods including administering to the individual a T-cell modulatory protein (TMP) and a PD-(L)1 CPI, (A) where the individual previously was determined to have progressive disease based on RECIST 1.1 criteria following (i) monotherapy with a PD-(L)1 CPI, or (ii) combination therapy comprising treatment with a PD-(L)1 CPI and a therapeutic agent other than the TMP (e.g., a chemotherapeutic agent, or (B) for individuals in any line of treatment where the individual has received a PD-L1 CPS of 0 or more than 0 to less than 1, e.g., individuals who (i) had no prior therapies following an initial diagnosis of HPV16+ cancer, (ii) had at least one prior therapy following an initial diagnosis of HPV16+ cancer and have been determined to have progressive disease, (iii) are receiving the combination as a neoadjuvant therapy for an HPV16+ cancer, (iv) are receiving the combination as a adjuvant therapy for an HPV16+ cancer, (v) are receiving the combination as a first line treatment following a diagnosis of recurrent and/or metastatic HPV16+ cancer, or (vi) are receiving the combination as a second line or beyond therapy following a diagnosis of recurrent and/or metastatic HPV16+ cancer and a diagnosis of progressive disease following a prior line of therapy. Such patients also may include individuals who, due to having a PD-L1 CPS below 1, might not have qualified for treatment with some PD-(L)1 CPIs such as pembrolizumab (either alone or in combination with one or more therapeutic agents such as one or more chemotherapeutic agent). Such HPV16-associated cancers include, e.g., cervical cancer, anal cancer, vulvar cancer, vaginal cancer, and head and neck cancers, e.g., squamous cell carcinoma, oropharyngeal cancer, nasopharyngeal cancer, hypopharyngeal cancer, and laryngeal cancer.

Individuals suitable for treatment include individuals whose cancer is associated with a PD-L1 CPS that is:
20 or less, e.g., 15 to 20, 10 to 15, or 1 to 5;
19 or less, e.g., 15 to 19, 10 to 15, or 1 to 5;
0 or more than 0 but less than 1;
1; and
more than 20, e.g., from 20 to 25, from 21 to 25, from 25 to 50, from 50 to 75, or greater than 75.

As noted above, a Combined Positive Score (CPS) is a measure of PD-L1 expression on cancer cells. Generally speaking, it is determined by an assay that measures the number of PD-L1-staining cells (tumor cells, lymphocytes, macrophages) relative to all viable tumor cells, multiplied by 100. See, e.g., Paintal and Brockstein (2020) Head and Neck Pathology 14:657. As noted above, “PD-L1 CPS” refers to a Combined Positive Score for PD-L1 expression received from any Approved Laboratory, and as noted above, an Approved Laboratory may or may not use an approved companion diagnostic assay to determine the PD-L1 CPS. Examples of assays to determine a PD-L1 CPS that might be used by an Approved Laboratory, include but are not limited to an approved PD-L1 IHC 22C3 pharmDx or other assay performed by an Approved Laboratory that employs a monoclonal mouse anti-PD-L1 antibody clone 22C3, or an approved PD-L1 IHC 28-8 pharmDx or other assay performed by an Approved Laboratory that employs a monoclonal mouse anti-PD-L1 antibody clone 28-8. Alternatively, an Approved Laboratory may use a different assay and/or a different antibody to determine the PD-L1 CPS.

### T-Cell Modulatory Polypeptides (TMPs)

A TMP suitable for use in a method of the present disclosure includes: i) a cancer-associated peptide (e.g., a cancer-associated peptide having a length of from 4 amino acids to 25 amino acids, e.g., from 8 amino acids to 14 amino acids or from 8 amino acids to 12 amino acids); ii) a first major histocompatibility complex (MHC) class I polypeptide that is a beta-2 microglobulin (B2M) polypeptide; iii) one or more immunomodulatory polypeptides; iv) a second MHC class I polypeptide that is heavy chain polypeptide; and v) a scaffold component. The TMP presents a peptide-MHC complex (pMHC) that can be specifically bound to a T-cell receptor (TCR) of a T cell. The pMHC is formed by the combination of the cancer-associated peptide, β2M polypeptide and the MHC class I heavy chain polypeptide. An “MHC class I heavy chain polypeptide” is sometimes referred to herein as a human leukocyte antigen (HLA) class I heavy chain polypeptide.

The scaffold component can be, e.g., an immunoglobulin (Ig) Fc polypeptide or a non-Ig Fc polypeptide. The TMP may comprise one or more independently selected peptide linkers between any two of the components of the TMP, e.g., between one or more of: i) the cancer-associated peptide and the β2M polypeptide; ii) the MHC class I heavy chain polypeptide and an immunomodulatory polypeptide; iii) the MHC class I heavy chain polypeptide and an Ig Fc polypeptide; iv) an Ig Fc polypeptide and an immunomodulatory polypeptide; and v) where the TMP comprises two immunomodulatory polypeptides, the two immunomodulatory polypeptides.

TMPs in which the Cancer-Associated Peptide is Part of a Fusion Protein

In some cases, the TMP is a single polypeptide chain, or is a protein that is a dimer comprising two single-chain TMP polypeptides. Examples of single-chain TMPs are described in WO 2022/197970 (Cue Biopharma, Inc.), the disclosure of which is expressly incorporated herein by reference.

In some cases, the TMP is a multimeric antigen-presenting polypeptide (MAPP) or a heterodimer or homodimer of MAPPs as described in WO 2021/242935 (Cue Biopharma, Inc.) or WO 2022/099157 (Cue Biopharma, Inc.), the disclosures of which are expressly incorporated by reference.

In some cases, the TMP is a heterodimer of two polypeptides or a protein that is a dimer of two heterodimers. Examples of heterodimeric TMPs and dimeric TMPs thereof are described in WO 2018/119114 and WO 2020/132136 (both to Cue Biopharma, Inc.), the disclosures of which as they pertain to such heterodimeric TMPs (referred to therein as “T-cell multimeric polypeptides”) and dimeric TMPs thereof is expressly incorporated by reference.

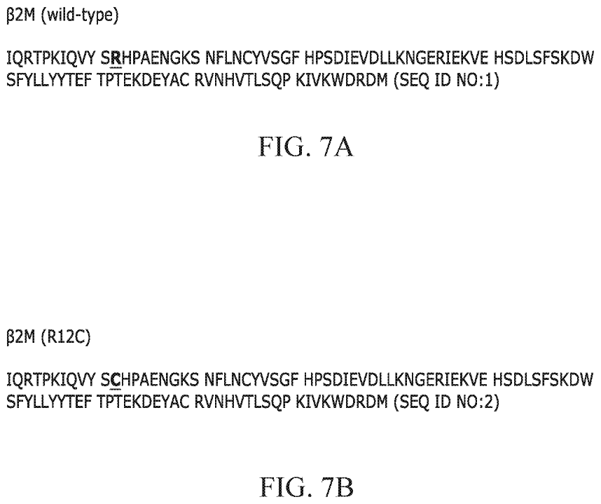

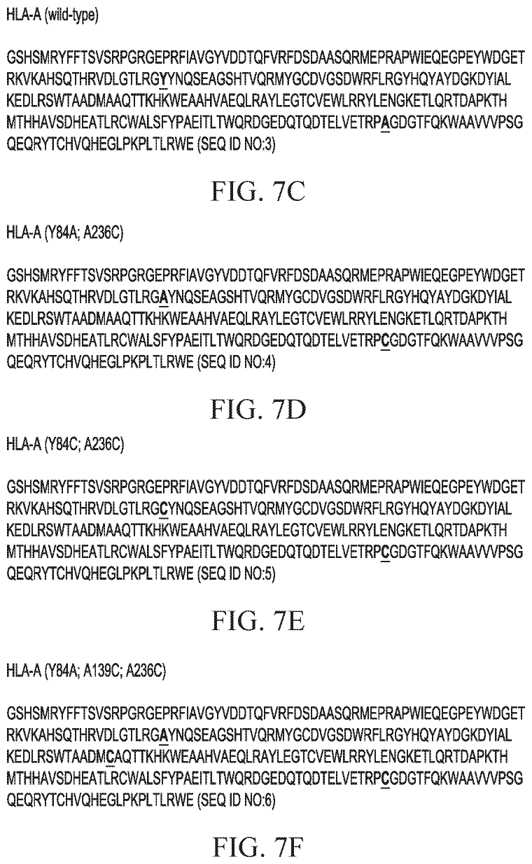

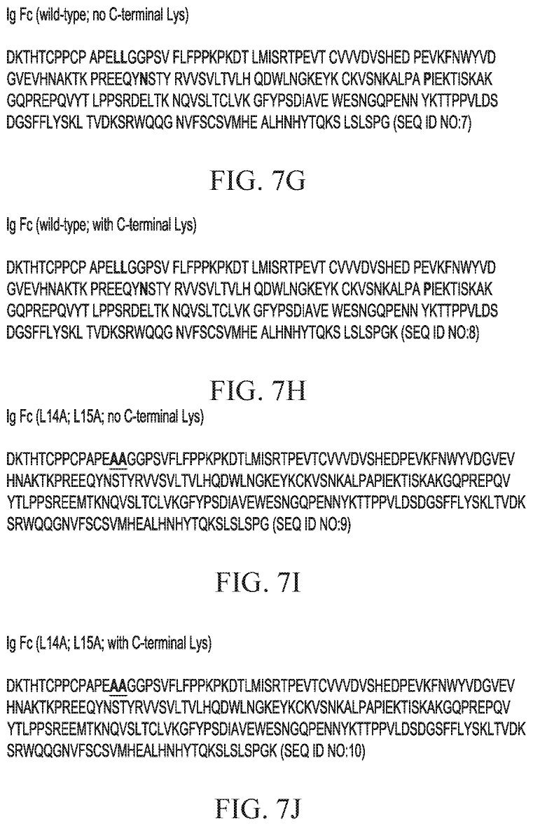

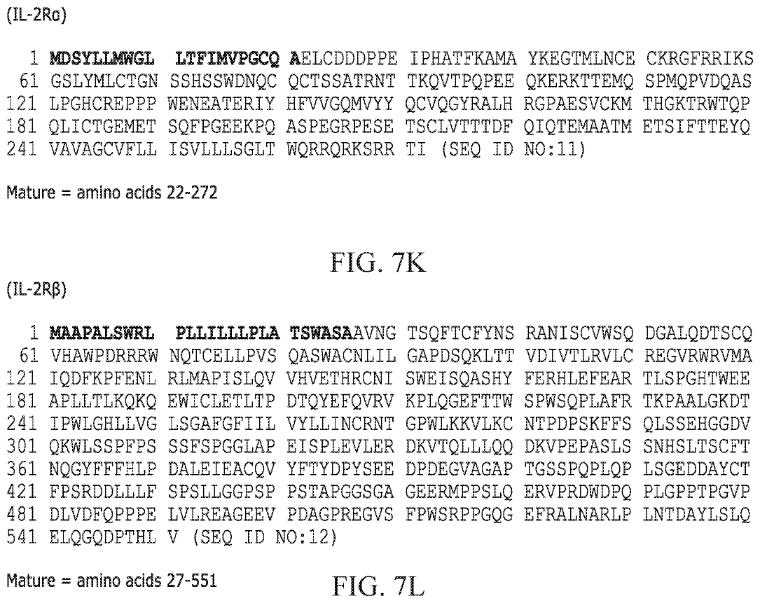

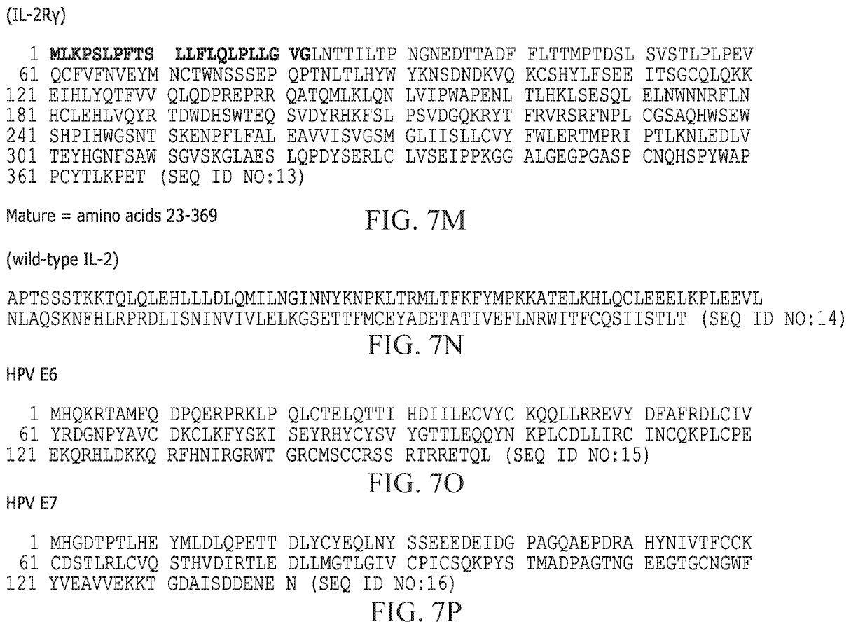

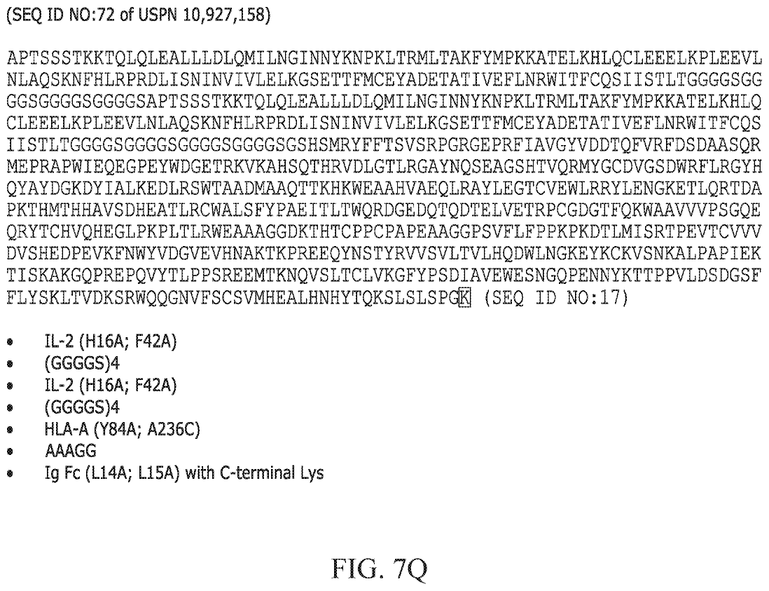

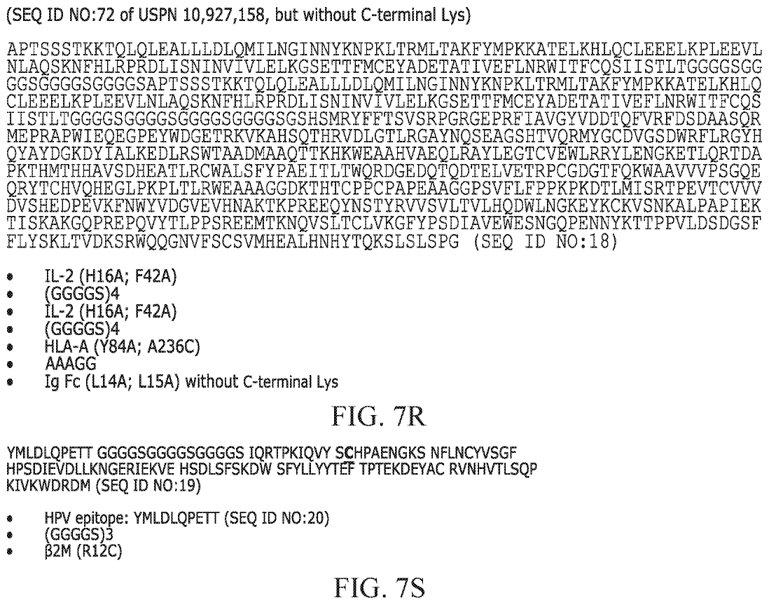

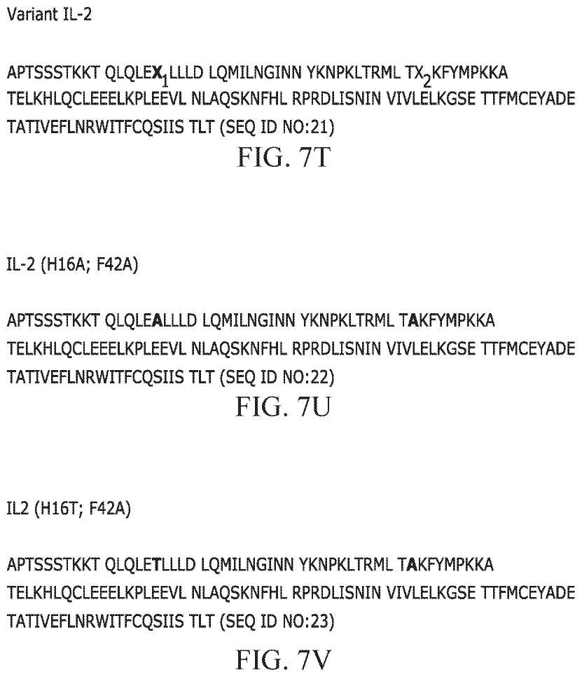

In some cases, the first polypeptide of a heterodimeric TMP (or of a dimeric TMP of two heterodimeric TMPs) comprises (i) a peptide from human papillomavirus (HPV), e.g., the HPV16 E7 (11-20) amino acid sequence YMLDLQPETT (SEQ ID NO:20), and (ii) a β2M polypeptide comprising an amino acid sequence as set forth in SEQ ID NO: 2 (depicted in FIG. 7B, where amino acid 12 is a Cys). The second polypeptide comprises (i) two copies of a variant IL-2 polypeptide in tandem, each copy comprising an amino acid sequence having at least about 95% percent amino acid sequence identity to the amino acid sequence set forth in SEQ ID NO:21 (depicted in FIG. 7T), wherein, as discussed below, the variant IL-2 polypeptides exhibits decreased or substantially no binding to IL-2Ra, thereby minimizing or substantially reducing the activation of Tregs by the IL-2 variant, and decreased binding to IL-2Rβ such that the IL-2 variant MOD exhibits an overall reduced affinity for IL-2R, (ii) an MHC class I heavy chain polypeptide, e.g., an MHC heavy chain polypeptide comprising an amino acid sequence having at least about 95% percent amino acid sequence identity to the amino acid sequence set forth in any one of SEQ ID Nos: 3-6 (depicted in FIGS. 7C-7F, and (iii) a Ig Fc polypeptide, e.g., an IgG1 Fc polypeptide having at least about 95% percent amino acid sequence identity to the amino acid sequence set forth in SEQ ID NO:9 or SEQ ID NO: 10 (depicted in FIGS. 7I and 7J), e.g., an Ig Fc that substantially does not induce cell lysis (as discussed below) through known mutations such as the LALA mutations discussed below. The first polypeptide typically comprises a peptide linker that joins the HPV peptide to the β2M polypeptide, and the second polypeptide typically may comprise one or more peptide linkers joining the components of second polypeptide.

The first and second polypeptides of each heterodimer are connected by at least one disulfide bond that spontaneously forms between the polypeptides. For example, the first and second polypeptides of a heterodimer may be joined by a disulfide bond that (i) connects a Cys residue in the β2M polypeptide to a Cys residue in the MHC class I heavy chain (e.g., a disulfide bond between the Cys residue at amino acid 12 of the β2M polypeptide and the Cys residue at amino acid 236 of the MHC class I heavy chain polypeptide). As another example, the first and second polypeptides of a heterodimer may be joined by a disulfide bond that connects (i) a Cys residue in the linker that joins the HPV peptide and the β2M polypeptide to (ii) a Cys residue in the MHC class I heavy chain polypeptide.

A disulfide bond formed between (i) a Cys amino acid in the linker between a cancer-associated peptide and a β2M polypeptide and (ii) a Cys in the MHC class I heavy chain polypeptide may be referred to as a “linker disulfide.” A disulfide bond formed between a Cys amino acid in β2M polypeptide and a Cys in the MHC class I heavy chain polypeptide may be referred to as a “body disulfide.” A heterodimeric or single-chain TMP may comprise either or both of a linker disulfide and a body disulfide. For example, the first and second polypeptides of a heterodimer may be joined by a first disulfide bond (a body disulfide) that connects a Cys residue in the β2M polypeptide to a Cys residue in the MHC class I heavy chain (e.g., a disulfide bond between the Cys residue at amino acid 12 of the β2M polypeptide and the Cys residue at amino acid 236 of the MHC class I heavy chain polypeptide), and a second disulfide bond (a linker disulfide) that connects (i) a Cys residue in the linker (e.g., a G2C linker as discussed below) that joins the HPV peptide to (ii) a Cys residue in the MHC class I heavy chain polypeptide. (Single-chain TMPs comprising a linker disulfide are discussed below.) The two heterodimers of the homodimer are joined to each other by one or more disulfide bonds (typically two disulfide bonds) that spontaneously form to join the Ig Fc polypeptide of one heterodimer to the Ig Fc polypeptide of the other heterodimer.

TMPs in which the Cancer-Associated Peptide is Chemically Conjugated

Unlike the above TMPs in which the cancer-associated peptide is part of a fusion protein with the β2M polypeptide (in the case of heterodimeric TMPs) or both the β2M polypeptide and the MHC class I polypeptide (in the case of single-chain TMPs), in some cases the TMP may comprise a cancer-associated polypeptide that is chemically conjugated to one of the MHC class polypeptides that comprise the pMHC.

In some cases, such TMPs comprising chemically conjugated cancer-associated peptides comprise heterodimeric polypeptides to which the cancer-associated peptide is chemically conjugated, or dimeric TMPs of such heterodimeric polypeptides. See, e.g., WO 2019/051127, WO 2020/132365, WO 2020/132366, and WO 2020/132368 (Cue Biopharma, Inc.), the disclosures of which are expressly incorporated by reference.

In some cases, such TMPs comprising chemically conjugated cancer-associated peptides comprise single-chain polypeptides to which the cancer-associated peptide is chemically conjugated, or dimer TMPs of such single-chain polypeptides. See, e.g., WO 2022/015880, WO 2022/099156 and WO 2022/137158 (Cue Biopharma, Inc.), the disclosures of which are expressly incorporated by reference. Such TMPs comprising chemically conjugated cancer-associated peptides may comprise one or more body disulfides but typically will not comprise a linker disulfide.

### HPV16 Cancer-Associated Peptides

As noted above, a TMP comprises an HPV16 cancer-associated peptide. As used herein, the term “peptide epitope” means a peptide that can be bound to MHC polypeptides (e.g., MHC class I polypeptides) and form a pMHC complex, such that the pMHC presents an epitope that can be specifically bound to a T-cell receptor (TCR) of a T cell.

An HPV16 cancer-associated peptide can have a length of at least 4 amino acids, e.g., from 4-20 aa (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa or 20 aa), including a range of from 6-15 aa, 8-12 aa, 8-14 aa, 8-16 aa, 8-10 aa, 9-11 aa, 5-10 aa, 10-15 aa, and 15-20 aa in length. In some cases, the peptide is 8, 9, 10, or 11 amino acids in length.

In some cases, an HPV peptide suitable for inclusion in a pMHC polypeptide can be a peptide of an HPV E6 polypeptide or an HPV E7 polypeptide. The HPV epitope can be an epitope of HPV of any of a variety of genotypes, including, e.g., HPV16, HPV18, HPV31, HPV33, HPV35, HPV39, HPV45, HPV51, HPV52, HPV56, HPV58, HPV59, HPV68, HPV73, or HPV82. In some cases, the peptide is an HPV E6 peptide. In some cases, the peptide is an HPV E7 peptide. An amino acid sequence of an HPV16 E6 polypeptide is presented in FIG. 7O (SEQ ID NO: 15). An amino acid sequence of an HPV16 E7 polypeptide is presented in FIG. 7P (SEQ ID NO: 16). In some cases, an HPV peptide is a peptide of from 4-20 aa, e.g., 6-15 aa, 8-12 aa, 8-10 aa, 9-11 aa, 5-10 aa, 10-15 aa, or 15-20 aa in length of an amino acid sequence having at least 90%, at least 95%, at least 97%, at least 98%, at least 99%, or 100%, amino acid sequence identity to the HPV E6 amino acid sequence depicted in FIG. 7O (and set forth in SEQ ID NO:15) or the HPV E7 amino acid sequence depicted in FIG. 7P (and set forth in SEQ ID NO: 16).

Examples of HPV E6 peptides suitable for inclusion in a TMP include, but are not limited to, E6 18-26 (KLPQLCTEL; SEQ ID NO:24); E6 26-34 (LQTTIHDII; SEQ ID NO:25); E6 49-57 (VYDFAFRDL; SEQ ID NO:26); E6 52-60 (FAFRDLCIV; SEQ ID NO:27); E6 75-83 (KFYSKISEY; SEQ ID NO:28); and E6 80-88 (ISEYRHYCY; SEQ ID NO:29).

Examples of HPV E7 peptides suitable for inclusion in a TMP include, but are not limited to, E7 7-15 (TLHEYMLDL; SEQ ID NO:30); E7 11-19 (YMLDLQPET; SEQ ID NO:31); E7 11-20 (YMLDLQPETT; SEQ ID NO:20); E7 44-52 (QAEPDRAHY; SEQ ID NO:32); E7 49-57 (RAHYNIVTF (SEQ ID NO:33); E7 61-69 (CDSTLRLCV; SEQ ID NO:34); and E7 67-76 (LCVQSTHVDI; SEQ ID NO:35); E7 82-90 (LLMGTLGIV; SEQ ID NO:36); E7 86-93 (TLGIVCPI; SEQ ID NO:37); and E7 92-93 (LLMGTLGIVCPI; SEQ ID NO:38).

In some cases, a suitable HPV peptide for inclusion in a TMP is an HPV E6 peptide that binds HLA-A24 (e.g., is an HLA-A2401-restricted epitope). Non-limiting examples include: VYDFAFRDL (SEQ ID NO:39 CYSLYGTTL (SEQ ID NO:40); EYRHYCYSL (SEQ ID NO:41); KLPQLCTEL (SEQ ID NO: 42); DPQERPRKL (SEQ ID NO:43); HYCYSLYGT (SEQ ID NO:44); DFAFRDLCI (SEQ ID NO: 45); LYGTTLEQQY (SEQ ID NO:46); HYCYSLYGTT (SEQ ID NO:47); EVYDFAFRDL (SEQ ID NO: 48); EYRHYCYSLY SEQ ID NO:49); VYDFAFRDLC (SEQ ID NO:50); YCYSIYGTTL (SEQ ID NO: 51); VYCKTVLEL (SEQ ID NO:52); VYGDTLEKL (SEQ ID NO:53); and LTNTGLYNLL (SEQ ID NO:54).

In some cases, a suitable HPV peptide for inclusion in a TMP is selected from the group consisting of: DLQPETTDL (SEQ ID NO:55); TLHEYMLDL (SEQ ID NO:56); TPTLHEYML (SEQ ID NO: 57); RAHYNIVTF (SEQ ID NO:58); GTLGIVCPI (SEQ ID NO:59); EPDRAHYNI (SEQ ID NO: 60); QLFLNTLSF (SEQ ID NO:61); FQQLFLNTL (SEQ ID NO:62); and AFQQLFLNTL (SEQ ID NO: 63).

In some cases, a suitable HPV peptide presents an HLA-A*2401-restricted epitope. Non-limiting examples of HPV peptides presenting an HLA-A*2401-restricted epitope include: VYDFAFRDL (SEQ ID NO: 64; RAHYNIVTF (SEQ ID NO:65; CDSTLRLCV (SEQ ID NO:66); and LCVQSTHVDI (SEQ ID NO: 67).

### β2M Polypeptides

The β2M polypeptide present in the TMP can comprise an amino acid sequence having at least 90%, at least 95%, at least 98%, at least 99%, or 100%, amino acid sequence identity to the following β2M amino acid sequence: IQRTPKIQVY SRHPAENGKS NFLNCYVSGF HPSDIEVDLLKNGERIEKVE HSDLSFSKDW SFYLLYYTEF TPTEKDEYAC RVNHVTLSQP KIVKWDRDM (SEQ ID NO:1) (depicted in FIG. 7A). In some cases, a β2M polypeptide comprises an amino acid sequence having at least 90%, at least 95%, at least 98%, at least 99%, or 100%, amino acid sequence identity to the following β2M amino acid sequence: IQRTPKIQVY SCHPAENGKS NFLNCYVSGF HPSDIEVDLLKNGERIEKVE HSDLSFSKDW SFYLLYYTEF TPTEKDEYAC RVNHVTLSQP KIVKWDRDM (SEQ ID NO:2) (depicted in FIG. 7B), where amino acid 12 is a Cys. In some cases, a β2M polypeptide comprises the amino acid sequence IQRTPKIQVY SCHPAENGKS NFLNCYVSGF HPSDIEVDLLKNGERIEKVE HSDLSFSKDW SFYLLYYTEF TPTEKDEYAC RVNHVTLSQP KIVKWDRDM (SEQ ID NO:2) (depicted in FIG. 7B).

### MHC Class I Heavy Chain Polypeptides

As noted above, a TMP comprises an MHC class I heavy chain polypeptide. In some cases, the MHC class I heavy chain is a heavy chain from an HLA-A, -B, -C, -E, -F, or -G allele. In some cases, the MHC class I heavy chain is a heavy chain from HLA-A*0101, A*0201, A*0301, A*1101, A*2301, A*2402, A*2407, A*3303, and/or A*3401. In some cases, the MHC class I heavy chain is a heavy chain from HLA-B*0702, B*0801, B*1502, B*3802, B*4001, B*4601, and/or B*5301. In some cases, the MHC class I heavy chain is a heavy chain from C*0102, C*0303, C*0304, C*0401, C*0602, C*0701, C*702, C*0801, and/or C*1502. In some cases, the MHC class I heavy chain is a heavy chain from HLA-E*0101 (HLA-E*01:01:01:01), HLA-E*01:03 (HLA-E*01:03:01:01), HLA-E*01:04, HLA-E*01:05, HLA-E*01:06, HLA-E*01:07, HLA-E*01:09, and HLA-E*01:10. Two HLA-E alleles that have a significant prevalence in humans are HLA-E*0101 (HLA-E*01:01:01:01) and HLA-E*01:03 (HLA-E*01:03:01:01).

In some cases, the TMP comprises an MHC class I HLA-A allele heavy chain. In some cases, the MHC class I heavy chain polypeptide comprises an amino acid sequence having at least 80%, at least 85%, at least 90%, at least 95%, at least 98%, at least 99%, or 100%, amino acid sequence identity to the following HLA-A heavy chain amino acid sequence:

(SEQ ID NO: 3)
GSHSMRYFFTSVSRPGRGEPRFIAVGYVDDTQFVRFDSDAASQRMEPRA
PWIEQEGPEYWDGETRKVKAHSQTHRVDLGTLRGYYNQSEAGSHTVQRM
YGCDVGSDWRFLRGYHQYAYDGKDYIALKEDLRSWTAADMAAQTTKHKW
EAAHVAEQLRAYLEGTCVEWLRRYLENGKETLQRTDAPKTHMTHHAVSD
HEATLRCWALSFYPAEITLTWQRDGEDQTQDTELVETRPAGDGTFQKWA
AVVVPSGQEQRYTCHVQHEGLPKPLTLRWE (depicted in
FIG. 7C).

In some cases, where the TMP comprises a heterodimer, the MHC class I heavy chain polypeptide of the second polypeptide comprises an Ala at position 84 and a Cys at position 236. Thus, e.g., in some cases, the MHC class I heavy chain polypeptide comprises an amino acid sequence having at least 80%, at least 85%, at least 90%, at least 95%, at least 98%, at least 99%, or 100%, amino acid sequence identity to the following HLA-A heavy chain amino acid sequence:

GSHSMRYFFTSVSRPGRGEPRFIAVGYVDDTQFVRFDSDAASQRMEPRAPWIEQEGPEY WDGETRKVKAHSQTHRVDLGTLRGAYNQSEAGSHTVQRMYGCDVGSDWRFLRGYHQYAYD GKDYIALKEDLRSWTAADMAAQTTKHKWEAAHVAEQLRAYLEGTCVEWLRRYLENGKETLQ RTDAPKTHMTHHAVSDHEATLRCWALSFYPAEITLTWQRDGEDQTQDTELVETRPCGDGTFQK WAAVVVPSGQEQRYTCHVQHEGLPKPLTLRWE (SEQ ID NO:4; see FIG. 7D), where amino acid 84 is an Ala and amino acid 236 is a Cys.

In some cases, where the TMP comprises a heterodimer, the MHC class I heavy chain polypeptide of the second polypeptide comprises a Cys at position 84 and a Cys at position 236. Thus, e.g., in some cases, the MHC class I heavy chain polypeptide comprises an amino acid sequence having at least 80%, at least 85%, at least 90%, at least 95%, at least 98%, at least 99%, or 100%, amino acid sequence identity to the following HLA-A heavy chain amino acid sequence:

GSHSMRYFFTSVSRPGRGEPRFIAVGYVDDTQFVRFDSDAASQRMEPRAPWIEQEGPEY WDGETRKVKAHSQTHRVDLGTLRGCYNQSEAGSHTVQRMYGCDVGSDWRFLRGYHQYAYD GKDYIALKEDLRSWTAADMAAQTTKHKWEAAHVAEQLRAYLEGTCVEWLRRYLENGKETLQ RTDAPKTHMTHHAVSDHEATLRCWALSFYPAEITLTWQRDGEDQTQDTELVETRPCGDGTFQK WAAVVVPSGQEQRYTCHVQHEGLPKPLTLRWE (SEQ ID NO:5; see FIG. 7E), where amino acid 84 is a Cys and amino acid 236 is a Cys.

In some cases, where the TMP comprises a heterodimer, the MHC class I heavy chain polypeptide of the second polypeptide comprises an Ala at position 84, a Cys at position 139, and a Cys at position 236. Thus, e.g., in some cases, the MHC class I heavy chain polypeptide comprises an amino acid sequence having at least 80%, at least 85%, at least 90%, at least 95%, at least 98%, at least 99%, or 100%, amino acid sequence identity to the following HLA-A heavy chain amino acid sequence:

GSHSMRYFFTSVSRPGRGEPRFIAVGYVDDTQFVRFDSDAASQRMEPRAPWIEQEGPEY WDGETRKVKAHSQTHRVDLGTLRGAYNQSEAGSHTVQRMYGCDVGSDWRFLRGYHQYAYD GKDYIALKEDLRSWTAADMCAQTTKHKWEAAHVAEQLRAYLEGTCVEWLRRYLENGKETLQ RTDAPKTHMTHHAVSDHEATLRCWALSFYPAEITLTWQRDGEDQTQDTELVETRPCGDGTFQK WAAVVVPSGQEQRYTCHVQHEGLPKPLTLRWE (SEQ ID NO:6; see FIG. 7F), where amino acid 84 is an Ala, amino acid 139 is a Cys, and amino acid 236 is a Cys.

Where the TMP is a single-chain TMP (or a dimeric TMP, e.g., homodimer, of single-chain TMPs), the TMP may comprise one or more spontaneously formed intrachain disulfide bonds, including either or both of a linker disulfide and one or more body disulfides. A linker disulfide may be formed between: (i) a Cys present in a peptide linker interposed between the HPV peptide and the β2M polypeptide (e.g., a G2C linker), and (ii) a Cys is substituted for one of the amino acids in the peptide binding groove of the MHC class I heavy chain polypeptide, e.g. a Cys substituted at position 84 or a Cys substituted at any one of amino acids 135-143 (e.g., at position 139), based on the numbering of the MHC class I heavy chain polypeptide depicted in FIG. 7F (SEQ ID NO:6). Where such a linker disulfide is formed with a Cys substituted at any one of amino acids 135-143, e.g., at position 139, then the amino acid at position 84 is other than a Cys, e.g., is Ala, Gly, Val or Tyr. Conversely, where such a linker disulfide is formed with a Cys substituted at position 84, then amino acids 135-143, e.g., at position 139, are other than Cys residues.

### Scaffold Polypeptides

As noted above, a TMP typically comprises a scaffold polypeptide such as an Ig Fc polypeptide or a non-Ig Fc polypeptide.

An Ig Fc polypeptide of a TMP can be a human IgG1 Fc, a human IgG2 Fc, a human IgG3 Fc, a human IgG4 Fc, etc., or a variant of a wild-type Ig Fc polypeptide. Variants include naturally occurring variants, non-naturally occurring variants, and combinations thereof. For example, the Ig Fc can be a variant of a Fc polypeptide such as a human IgG1 Fc, which variant has a reduced or substantially eliminated ability to effect complement-dependent cytotoxicity (CDC) or antibody-dependent cell-mediated cytotoxicity (ADCC).

In some cases, e.g., where TMP comprises a heterodimer of first and second polypeptides, the Ig Fc may be at the C-terminus of the second polypeptide. In such cases, the Ig Fc polypeptide optionally does not comprise a C-terminal Lys.

In some cases, the Fc polypeptide present in a TMP is an IgG1 Fc polypeptide, or a variant of an IgG1 Fc polypeptide. For example, in some cases, the IgG1 Fc polypeptide present in a TMP comprises an amino acid sequence having at least 80%, at least 85%, at least 90%, at least 95%, at least 98%, at least 99%, or 100%, amino acid sequence identity to the following human IgG1 Fc polypeptide amino acid sequence: DKTHTCPPCP APELLGGPSV FLFPPKPKDT LMISRTPEVT CVVVDVSHED PEVKFNWYVD GVEVHNAKTK PREEQYNSTY RVVSVLTVLH QDWLNGKEYK CKVSNKALPA PIEKTISKAK GQPREPQVYT LPPSRDELTK NQVSLTCLVK GFYPSDIAVE WESNGQPENN YKTTPPVLDS DGSFFLYSKL TVDKSRWQQG NVFSCSVMHE ALHNHYTQKS LSLSPG (SEQ ID NO:7) (depicted in FIG. 7G). Such IgG1 Fc polypeptides may or may not comprise a C-terminal lysine.

In some cases, the Fc polypeptide present in a TMP comprises an amino acid sequence having at least 80%, at least 85%, at least 90%, at least 95%, at least 98%, at least 99%, or 100%, amino acid sequence identity to the following human IgG1 Fc polypeptide amino acid sequence: DKTHTCPPCP APELLGGPSV FLFPPKPKDT LMISRTPEVT CVVVDVSHED PEVKFNWYVD GVEVHNAKTK PREEQYNSTY RVVSVLTVLH QDWLNGKEYK CKVSNKALPA PIEKTISKAK GQPREPQVYT LPPSRDELTK NQVSLTCLVK GFYPSDIAVE WESNGQPENN YKTTPPVLDS DGSFFLYSKL TVDKSRWQQG NVFSCSVMHE ALHNHYTQKS LSLSPGK (SEQ ID NO:8; depicted in FIG. 7H).

In some cases, the Ig Fc polypeptide present in a TMP comprises an amino acid sequence having at least 80%, at least 85%, at least 90%, at least 95%, at least 98%, at least 99%, or 100%, amino acid sequence identity to the Ig Fc polypeptide amino acid sequence set forth in SEQ ID NO:9 or SEQ ID NO: 10 (and depicted in FIG. 7I or FIG. 7J), where amino acid 14 is an Ala and amino acid 15 is an Ala. (As discussed below, substitutions of Ala for Leu at amino acids 14 and 15 are known as the “LALA” mutations.) Thus, in some cases, the Ig Fc polypeptide present in a TMP comprises an amino acid sequence having at least 80%, at least 85%, at least 90%, at least 95%, at least 98%, at least 99%, or 100%, amino acid sequence identity to the following Ig Fc amino acid sequence: DKTHTCPPCPAPEAAGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKAKGQPREPQ VYTLPPSREEMTKNQVSLTCLVKGFYPSDIAVEWESNGQPENNYKTTPPVLDSDGSFFLYSKLT VDKSRWQQGNVFSCSVMHEALHNHYTQKSLSLSPG (SEQ ID NO:9; see FIG. 7I), where amino acid 14 is an Ala and amino acid 15 is an Ala, and where the Ig Fc omits a C-terminal Lys.

In some cases, the Ig Fc polypeptide present in a TMP comprises an amino acid sequence having at least 80%, at least 85%, at least 90%, at least 95%, at least 98%, at least 99%, or 100%, amino acid sequence identity to the following Ig Fc amino acid sequence: DKTHTCPPCPAPEAAGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKAKGQPREPQ VYTLPPSREEMTKNQVSLTCLVKGFYPSDIAVEWESNGQPENNYKTTPPVLDSDGSFFLYSKLT VDKSRWQQGNVFSCSVMHEALHNHYTQKSLSLSPGK (SEQ ID NO: 10; see FIG. 7J), where amino acid 14 is an Ala and amino acid 15 is an Ala, and where the Ig Fc comprises a C-terminal Lys.

### Immunomodulatory Polypeptides

As noted above, a TMP comprises one or more immunomodulatory polypeptides (“MODs”). In some cases, a MOD present in the TMP is a wild-type (“wt”) MOD. In other cases, a MOD present in a TMP is a variant of a wt MOD that has reduced affinity for a co-MOD compared to the affinity of a corresponding wild-type MOD for the co-MOD. Suitable MODs that exhibit reduced affinity for a co-MOD can have from 1 amino acid (aa) to 20 aa differences from a wild-type MOD. For example, in some cases, a variant MOD present in a TMP differs in amino acid sequence by 1 aa, 2 aa, 3 aa, 4 aa, 5 aa, 6 aa, 7 aa, 8 aa, 9 aa, or 10 aa, from a corresponding wild-type MOD. As another example, in some cases, a variant MOD present in a TMP differs in amino acid sequence by 11 aa, 12 aa, 13 aa, 14 aa, 15 aa, 16 aa, 17 aa, 18 aa, 19 aa, or 20 aa, from a corresponding wild-type MOD.

As discussed above, a MOD may comprise a variant of a wt MOD that may exhibit reduced binding to its co-MOD, including e.g., reduced binding to one or more chains or domains of the co-MOD. For example, a variant MOD present in a TMP may bind its co-MOD with an affinity that it at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the affinity of a corresponding wild-type MOD for the co-MOD.

Exemplary pairs of MODs and their co-MODs include, but are not limited to those set out in Table 1, below:

TABLE 1

| Immunomodulatory | |
|---|---|
| Polypeptide (MOD) | Co-MOD |
| 4-1BBL | 4-1BB |
| PD-L1 | PD-1 |
| IL-2 | IL-2 receptor |
| CD80 | CD28 |
| CD86 | CD28 |
| OX40L (CD252) | OX40 (CD134) |
| Fas ligand | Fas |
| ICOS-L | ICOS |
| ICAM | LFA-1 |
| CD30L | CD30 |
| CD40 | CD40L |
| CD83 | CD83L |
| HVEM (CD270) | CD160 |
| JAG1 (CD339) | Notch |
| JAG1 | CD46 |
| CD80 | CTLA4 |
| CD86 | CTLA4 |
| CD70 | CD27 |
| TGFβ | TGFβ receptor |

Wild-type immunomodulatory polypeptides and variants, including reduced affinity variants, such as PD-L1, CD80, CD86, 4-1BBL and IL-2 are described in the published literature, e.g., published PCT application WO2020132138A1 and WO2019/051091 (Cue Biopharma, Inc.), the disclosures of which as they pertain to MODs and specific variant MODs of PD-L1, CD80, CD86, 4-1BBL, IL-2 are expressly incorporated herein by reference, including specifically paragraphs [00260]-[00455] of WO2020132138A1 and paragraphs [00157]-[00352] of WO2019/051091.

Of interest are MODs that are variants of the cytokine IL-2. Wild-type IL-2 binds to IL-2 receptor (IL-2R) on the surface of a T cell. Wild-type IL-2 has a strong affinity for IL-2R and will bind to activate most or substantially all CD8+ T cells. For this reason, synthetic forms of wild type IL-2 such as the drug Aldesleukin (trade name Proleukin®) are known to have severe side-effects when administered to humans for the treatment of cancer because the IL-2 indiscriminately activates both target and non-target T cells.

An IL-2 receptor is in some cases a heterotrimeric polypeptide comprising an alpha chain (IL-2Rα; also referred to as CD25), a beta chain (IL-2Rβ; also referred to as CD122; and a gamma chain (IL-2Rγ; also referred to as CD132). Amino acid sequences of human IL-2, human IL-2Rα, IL2Rβ, and IL-2Rγ are known. See, e.g., published PCT applications WO2020132138A1 and WO2019/051091, discussed above. For example, a wild-type IL-2 polypeptide can have the amino acid sequence depicted in FIG. 7N (SEQ ID NO:14). Amino acid sequences of human IL-2Rα, human IL-2Rβ, and human IL-2Rγ are depicted in FIGS. 7K, 7L, and 7M, respectively, where the mature form of IL-2Ra is amino acids 22-272 of the amino acid sequence depicted in FIG. 7K, the mature form of IL-2Rβ is amino acids 27-551 of the amino acid sequence depicted in FIG. 7L, and the mature form of IL-2Rγ is amino acids 23-369 of the amino acid sequence depicted in FIG. 7M.

In some cases, an IL-2 variant MOD of this disclosure exhibits decreased binding to IL-2Rα, thereby minimizing or substantially reducing the activation of Tregs by the IL-2 variant. Alternatively, or additionally, in some cases, an IL-2 variant MOD of this disclosure exhibits decreased binding to IL-2RB and/or IL-2Rγ such that the IL-2 variant MOD exhibits an overall reduced affinity for IL-2R. In some cases, an IL-2 variant MOD of this disclosure exhibits both properties, i.e., it exhibits decreased or substantially no binding to IL-2Rα, and also exhibits decreased binding to IL-2Rβ and/or IL-2Rγ such that the IL-2 variant polypeptide exhibits an overall reduced affinity for IL-2R. For example, IL-2 variants having substitutions at H16 and F42 have shown decreased binding to IL-2Ra and IL-2Rβ. See, Quayle et al., Clin Cancer Res; 26 (8) Apr. 15, 2020, which discloses that the binding affinity of an IL-2 polypeptide with H16A and F42A substitutions for human IL-2Ra and IL-2Rβ was decreased 110- and 3-fold, respectively, compared with wild-type IL2 binding, predominantly due to a faster off-rate for each of these interactions. TMPs comprising such variants, including variants that exhibit decreased binding to IL-2Ra and IL-2Rβ, have shown the ability to preferentially bind to and activate IL-2 receptors on T cells that contain the target TCR that is specific for the epitope presented by the pMHC of the TMP, and are thus less likely to deliver IL-2 to non-target T cells, i.e., T cells that do not contain a TCR that specifically binds the epitope presented by the pMHC of the TMP. That is, the binding of the IL-2 variant MOD to its costimulatory polypeptide on the T cell is substantially driven by the binding of the MHC-epitope moiety rather than by the binding of the variant IL-2 polypeptide to IL-2R receptors on the T cell.

Suitable IL-2 variant MODs thus include a polypeptide that comprises an amino acid sequence having at least 90%, at least 95%, at least 97%, at least 98%, or at least 99% amino acid sequence identity to the wild-type IL-2 amino acid sequence depicted in FIG. 7N (SEQ ID NO:14); and that have one or more amino acid differences from the wild-type IL-2 amino acid sequence depicted in FIG. 7N. In some cases, such a variant IL-2 polypeptide of this disclosure exhibits reduced binding affinity to IL-2R, compared to the binding affinity of an IL-2 polypeptide comprising the wild-type IL-2 amino acid sequence depicted in FIG. 7N. For example, in some cases, a variant IL-2 polypeptide binds IL-2R with a binding affinity that Is at least 10% less, at least 15% less, at least 20% less, at least 25%, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of an IL-2 polypeptide comprising the wild-type IL-2 amino acid sequence depicted in FIG. 7N for an IL-2R (e.g., an IL-2R comprising polypeptides comprising the amino acid sequences depicted in FIGS. 7K-7M, e.g., the mature forms of the amino acid sequences depicted in FIGS. 7K-7M), when assayed under the same conditions.

Some exemplary combinations of mutations that reduce binding of an IL-2 variant polypeptide to IL-2Ra and IL-2Rβ include the following from Table 2:

TABLE 2

| Mutation(s) to | Mutation(s) to | |
|---|---|---|
| decrease binding | decrease binding | |
| to IL-2Rα | to IL-2Rβ | Exemplary combinations |
| R38 with any | E15 with any | E15A with R38A, R38D or R38E |
| amino acid other | amino acid other | |
| than Arg, e.g., Ala, | than Glu | |
| Asp, Glu | | |
| R38 with any | H16, with any | H16A with R38A, R38D or R38E |
| amino acid other | amino acid other | H16T with R38A, R38D or R38E |
| than Arg, e.g., Ala, | than His, e.g., Ala, | H16E with R38A, R38D or R38E |
| Asp, Glu | Glu, Thr, or Asp, | H16D with R38A, R38D or R38E |
| R38 with any | D84, with any | D84H with R38A, R38D or R38E |
| amino acid other | amino acid other | D84K with R38A, R38D or R38E |
| than Arg, e.g., Ala, | than Asp, e.g., His, | D84R with R38A, R38D or R38E |
| Asp, Glu | Lys or Arg | |
| R38 with any | N88, with any | R38a with N88S, N88A, N88G, N88R, N88T, or N88D |
| amino acid other | amino acid other | R38D with N88S, N88A, N88G, N88R, N88T, or N88D |
| than Arg, e.g., Ala, | than Asn, e.g., Ser, | R38E with N88S, N88A, N88G, N88R, N88T, or N88D |
| Asp, Glu | Ala, Gly, Arg, Thr | |
| | or Asp | |
| R38 with any | V91 with any | R38A with V91E, V91A or V91T |
| amino acid other | amino acid other | R38D with V91E, V91A or V91T |
| than Arg, e.g., Ala, | than Val, e.g., Glu, | R38E with V91E, V91A or V91T |
| Asp, Glu | Ala or Thr | |
| R38 with any | I92 with any amino | R38A, I92A |
| amino acid other | acid other than Ile, | R38D, I92A |
| than Arg, e.g., Ala, | e.g., Ala | R38E, I92A |
| Asp, Glu | | |
| F42, with any | E15 with any | E15A, F42A |
| amino acid other | amino acid other | E15A, F42K |
| than Phe, e.g., Ala | than Glu | |
| or Lys, as well as | | |
| Met, Pro, Ser, Thr, | | |
| Trp, Tyr, and Val | | |
| F42, with any | H16, with any | H16A, F42A; H16T, F42A; |
| amino acid other | amino acid other | H16E, F42A; H16D, F42A |
| than Phe, e.g., Ala | than His, e.g., Ala, | H16A, F42K; H16T, F42K; |
| or Lys, as well as | Glu, Thr, or Asp, | H16E, F42K; H16D, F42K |
| Met, Pro, Ser, Thr, | | |
| Trp, Tyr, and Val | | |
| F42, with any | D84, with any | F42A with D84H, D84K or D84R |
| amino acid other | amino acid other | F42K with D84H, D84K or D84R |
| than Phe, e.g., Ala | than Asp, e.g., His, | |
| or Lys, as well as | Lys or Arg | |
| Met, Pro, Ser, Thr, | | |
| Trp, Tyr, and Val | | |
| F42, with any | N88, with any | F42A with N88S, N88A, N88G, N88R, N88T, or N88D |
| amino acid other | amino acid other | F42K with N88S, N88A, N88G, N88R, N88T, or N88D |
| than Phe, e.g., Ala | than Asn, e.g., Ser, | |
| or Lys, as well as | Ala, Gly, Arg, Thr | |
| Met, Pro, Ser, Thr, | or Asp | |
| Trp, Tyr, and Val | | |
| F42, with any | V91 with any | F42A with V91E, V91A, or V91T |
| amino acid other | amino acid other | F42K with V91E, V91A, or V91T |
| than Phe, e.g., Ala | than Val, e.g., Glu, | |
| or Lys, as well as | Ala or Thr | |
| Met, Pro, Ser, Thr, | | |
| Trp, Tyr, and Val | | |
| F42, with any | I92 with any amino | F42A with I92A |
| amino acid other | acid other than Ile, | F42K with I92A |
| than Phe, e.g., Ala | e.g., Ala | |
| or Lys, as well as | | |
| Met, Pro, Ser, Thr, | | |
| Trp, Tyr, and Val | | |
| K43, with any | E15 with any | E15A, K43E |
| amino acid other | amino acid other | |
| than Lys, e.g., Glu | than Glu | |
| K43, with any | H16, with any | H16A, K43E; H16T, K43E; |
| amino acid other | amino acid other | H16E, K43E; H16D, K43E |
| than Lys, e.g., Glu | than His, e.g., Ala, | |
| | Glu, Thr, or Asp, | |
| K43, with any | D84, with any | K43E with D84H, D84K or D84R |
| amino acid other | amino acid other | |
| than Lys, e.g., Glu | than Asp, e.g., His, | |
| | Lys or Arg | |
| K43, with any | N88, with any | K43E with N88S, N88A, N88G, N88R, N88T, or N88D |
| amino acid other | amino acid other | |
| than Lys, e.g., Glu | than Asn, e.g., Ser, | |
| | Ala, Gly, Arg, Thr | |
| | or Asp | |
| K43, with any | V91 with any | K43E with V91E, V91A, or V91T |
| amino acid other | amino acid other | |
| than Lys, e.g., Glu | than Val, e.g., Glu, | |
| | Ala or Thr | |
| K43, with any | I92 with any amino | K43E, I92A |
| amino acid other | acid other than Ile, | |
| than Lys, e.g., Glu | e.g., Ala | |
| E62, with any | E15 with any | E15A, E62Q |
| amino acid other | amino acid other | |
| than Glu, e.g., Gln | than Glu | |
| E62, with any | H16, with any | H16A, E62Q; H16T, E62Q; |
| amino acid other | amino acid other | H16E, E62Q; H16D, E62Q |
| than Glu, e.g., Gln | than His, e.g., Ala, | |
| | Glu, Thr, or Asp, | |
| E62, with any | D84, with any | E62Q with D84H, D84K or D84R |
| amino acid other | amino acid other | |
| than Glu, e.g., Gln | than Asp, e.g., His, | |
| | Lys or Arg | |
| E62, with any | N88, with any | E62Q with N88S, N88A, N88G, N88R, N88T, or N88D |
| amino acid other | amino acid other | |
| than Glu, e.g., Gln | than Asn, e.g., Ser, | |
| | Ala, Gly, Arg, Thr | |
| | or Asp | |
| E62, with any | V91 with any | E62Q with V91E, V91A, or V91T |
| amino acid other | amino acid other | |
| than Glu, e.g., Gln | than Val, e.g., Glu, | |
| | Ala or Thr | |
| E62, with any | I92 with any amino | E62Q, I92A |
| amino acid other | acid other than Ile, | |
| than Glu, e.g., Gln | e.g., Ala | |
| F42, with any | E15 with any | E15A, F42A with D84H, D84K or D84R |
| amino acid other | amino acid other | E15A, F42K with D84H, D84K or D84R |
| than Phe, e.g., Ala | than Glu | |
| or Lys, as well as | D84, with any | |
| Met, Pro, Ser, Thr, | amino acid other | |
| Trp, Tyr, and Val | than Asp, e.g., His, | |
| | Lys and Arg | |
| F42, with any | E15 with any | E15A, F42A with N88S, N88A, N88G, N88R, N88T, or |
| amino acid other | amino acid other | N88D |
| than Phe, e.g., Ala | than Glu | E15A, F42K with N88S, N88A, N88G, N88R, N88T, or |
| or Lys, as well as | N88, with any | N88D |
| Met, Pro, Ser, Thr, | amino acid other | |
| Trp, Tyr, and Val | than Asn, e.g., Ser, | |
| | Ala, Gly, Arg, Thr, | |
| | and Asp | |
| F42, with any | E15 with any | E15A, F42A with V91E, V91A, or V91T |
| amino acid other | amino acid other | E15A, F42K with V91E, V91A, or V91T |
| than Phe, e.g., Ala | than Glu | |
| or Lys, as well as | V91 with any | |
| Met, Pro, Ser, Thr, | amino acid other | |
| Trp, Tyr, and Val | than Val, e.g., Glu, | |
| | Ala or Thr | |
| F42, with any | E15 with any | E15A, F42A with I92A |
| amino acid other | amino acid other | E15A, F42K with I92A |
| than Phe, e.g., Ala | than Glu | |
| or Lys, as well as | I92 with any amino | |
| Met, Pro, Ser, Thr, | acid other than Ile, | |
| Trp, Tyr, and Val | e.g., Ala | |
| F42, with any | H16, with any | H16A, F42A with D84H, D84K or D84R |
| amino acid other | amino acid other | H16A, F42K with D84H, D84K or D84R |
| than Phe, e.g., Ala | than His, e.g., Ala, | H16T, F42A with D84H, D84K or D84R |
| or Lys, as well as | Glu, Thr, or Asp | H16T, F42K with D84H, D84K or D84R |
| Met, Pro, Ser, Thr, | D84, with any | H16E, F42A with D84H, D84K or D84R |
| Trp, Tyr, and Val | amino acid other | H16E, F42K with D84H, D84K or D84R |
| | than Asp, e.g., His, | H16D, F42A with D84H, D84K or D84R |
| | Lys and Arg | H16D, F42K with D84H, D84K or D84R |
| F42, with any | H16, with any | H16A, F42A with N88S, N88A, N88G, N88R, N88T, or |
| amino acid other | amino acid other | N88D |
| than Phe, e.g., Ala | than His, e.g., Ala, | H16A, F42K with N88S, N88A, N88G, N88R, N88T, or |
| or Lys, as well as | Glu, Thr, or Asp | N88D |
| Met, Pro, Ser, Thr, | N88, with any | H16T, F42A with N88S, N88A, N88G, N88R, N88T, or |
| Trp, Tyr, and Val | amino acid other | N88D |
| | than Asn, e.g., Ser, | H16T, F42K with N88S, N88A, N88G, N88R, N88T, or |
| | Ala, Gly, Arg, Thr, | N88D |
| | and Asp | H16E, F42A with N88S, N88A, N88G, N88R, N88T, or |
| | | N88D |
| | | H16E, F42K with N88S, N88A, N88G, N88R, N88T, or |
| | | N88D |
| | | H16D, F42A with N88S, N88A, N88G, N88R, N88T, or |
| | | N88D |
| | | H16D, F42K with N88S, N88A, N88G, N88R, N88T, or |
| | | N88D |
| F42, with any | H16, with any | H16A, F42A with V91E, V91A, or V91T |
| amino acid other | amino acid other | H16A, F42K with V91E, V91A, or V91T |
| than Phe, e.g., Ala | than His, e.g., Ala, | H16T, F42A with V91E, V91A, or V91T |
| or Lys, as well as | Glu, Thr, or Asp | H16T, F42K with V91E, V91A, or V91T |
| Met, Pro, Ser, Thr, | V91 with any | H16E, F42A with V91E, V91A, or V91T |
| Trp, Tyr, and Val | amino acid other | H16E, F42K with V91E, V91A, or V91T |
| | than Val, e.g., Glu, | H16D, F42A with V91E, V91A, or V91T |
| | Ala or Thr | H16D, F42K with V91E, V91A, or V91T |
| F42, with any | H16, with any | H16A, F42A with I92A |
| amino acid other | amino acid other | H16A, F42K with I92A |
| than Phe, e.g., Ala | than His, e.g., Ala, | H16T, F42A with I92A |
| or Lys, as well as | Glu, Thr, or Asp | H16T, F42K with I92A |
| Met, Pro, Ser, Thr, | I92 with any amino | H16E, F42A with I92A |
| Trp, Tyr, and Val | acid other than Ile, | H16E, F42K with I92A |
| | e.g., Ala | H16D, F42A with I92A |
| | | H16D, F42K with I92A |

In some cases, a suitable variant IL-2 polypeptide comprises an amino acid sequence having at least 90%, at least 95%, at least 97%, at least 98%, at least 99%, or 100% amino acid sequence identity to the amino acid sequence: APTSSSTKKT QLQLEX₁LLLD LQMILNGINN YKNPKLTRML TX₂KFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:21; depicted in FIG. 7T), where X₁ is an amino acid other than His, and where X₂ is an amino acid other than Phe. In some cases, a suitable variant IL-2 polypeptide comprises an amino acid sequence having at least 90%, at least 95%, at least 97%, at least 98%, at least 99%, or 100% amino acid sequence identity to the amino acid sequence: APTSSSTKKT QLQLEX₁LLLD LQMILNGINN YKNPKLTRML TX₂KFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:21; depicted in FIG. 7T), where: i) X₁ is Ala, Arg, Asn, Asp, Cys, Gln, Glu, Gly, Ile, Leu, Lys, Met, Phe, Pro, Ser, Thr, Trp, Tyr, or Val; and ii) X₂ is Ala, Arg, Asn, Asp, Cys, Gln, Glu, Gly, His, Ile, Leu, Lys, Met, Pro, Ser, Thr, Trp, Tyr, or Val. In some cases, X₁ is Ala and X₂ is Ala. In some cases, X₁ is Thr and X₂ is Ala. In some cases, X₁ is Asp and X₂ is Ala. In some cases, X₁ is Glu and X₂ is Ala.

In some cases, a suitable variant IL-2 polypeptide comprises an amino acid sequence having at least 90%, at least 95%, at least 97%, at least 98%, at least 99%, or 100% amino acid sequence identity to the amino acid sequence: APTSSSTKKT QLQLEALLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:22; depicted in FIG. 7U), i.e., the variant IL-2 polypeptide has the amino acid sequence of wild-type IL-2 but with H16A and F42A substitutions (shown in bold). Alternatively, the foregoing sequence, but with substitutions other than Ala at H16 and/or F42 may be employed, e.g., H16T may be employed instead of H16A. In some cases, a variant IL-2 polypeptide present in a TMP comprises the amino acid sequence: APTSSSTKKT QLQLEALLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:22). In some cases, a variant IL-2 polypeptide present in a TMP comprises the amino acid sequence: APTSSSTKKT QLQLETLLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:23; depicted in FIG. 7V). In some cases, a TMP comprises two copies of such a variant IL-2 polypeptide. Where a TMP comprises two copies of a variant IL-2 polypeptide, in some cases, the two copies are in tandem. Where a TMP comprises two copies of a variant IL-2 polypeptide, and where the two copies are in tandem, in some cases, the TMP comprises a peptide linker between the two copies.

### Linkers

A TMP of the present disclosure can include one or more peptide linkers interposed between any two components of the TMP. For example, one or more linkers may be interposed between one or more of: i) a cancer-associated peptide and a β2M polypeptide; ii) an MHC class I heavy chain polypeptide and an Ig Fc polypeptide; iii) an MHC class I heavy chain polypeptide and an immunomodulatory polypeptide; iv) an Ig Fc polypeptide and an immunomodulatory polypeptide; and v) where a TMP comprises two immunomodulatory polypeptides in tandem, between the two immunomodulatory polypeptides. As used herein, the phrase “an optional peptide linker between any two of the components of a TMP” refers to a peptide linker between any two adjacent polypeptides within the TMP. For example, as used herein, the phrase “an optional peptide linker between any two of the components of a TMP” refers to a peptide linker between one or more of: i) a cancer-associated peptide and a β2M polypeptide; ii) a β2M polypeptide and an MHC class I heavy chain polypeptide; iii) an MHC class I heavy chain polypeptide and an Ig Fc polypeptide; iv) an MHC class I heavy chain polypeptide and a MOD; v) an Ig Fc polypeptide and a MOD; and vi) between adjacent MODs when there are two or more MODs in tandem.

Linkers may be a flexible peptide linker, including a short flexible peptide linker, or a rigid peptide linker. Such linkers are disclosed in WO 2022/197970 (Cue Biopharma, Inc.), the disclosure of which as it pertains to such peptides is expressly incorporated herein by reference, including specifically, but not limited to, paragraphs [00188]-[00196].

As noted above, the linker between a cancer-associated peptide and a β2M polypeptide may comprise a single Cys residue that can form a disulfide bond with a Cys in the MHC class I heavy chain polypeptide. Such Cys-containing linkers are disclosed in WO 2022/197970 (Cue Biopharma, Inc.), the disclosure of which as it pertains to such peptide linkers is expressly incorporated herein by reference, including specifically, but not limited to, paragraphs [00202]-[00207]. Where the TMP comprises a heterodimer, the Cys in the linker can form a disulfide bond with a Cys in the MHC class I heavy chain polypeptide in the second polypeptide. Where the TMP comprises a single-chain polypeptide, the Cys in the linker can form an intrachain disulfide bond with a Cys in the MHC class I heavy chain polypeptide, e.g., with a Cys at position 84 or a Cys at one of positions 135-143, e.g., at position 139.

### Heterodimeric TMPs

As noted above, in some cases, the TMP is a heterodimer, or is a TMP comprising a dimer (a homodimer or a heterodimer) of two heterodimers. The components of the TMP can be arranged in any of a variety of configurations.

For example, in some cases, a TMP comprises a heterodimer, wherein the heterodimer comprises: a) a first polypeptide comprising: i) a cancer-associated peptide; ii) an optional peptide linker; and iii) a β2M polypeptide; and b) a second polypeptide comprising: i) one or more immunomodulatory polypeptides (where, if the TMP comprises two MODs in tandem, the MODs may be separated by a peptide linker); ii) an optional peptide linker; iii) an MHC class I heavy chain polypeptide; iv) an optional peptide linker; and v) an Ig Fc polypeptide. For example, in some cases, a TMP comprises a heterodimer, wherein the heterodimer comprises: a) a first polypeptide comprising, in order from N-terminus to C-terminus: i) a cancer-associated peptide; ii) a peptide linker; and iii) a β2M polypeptide; and b) a second polypeptide comprising, in order from N-terminus to C-terminus: i) one or more MODs (where, if the TMP comprises two MODs in tandem, the MODs may be separated by a peptide linker); ii) an optional peptide linker; iii) an MHC class I heavy chain polypeptide; iv) an optional linker; and v) an Ig Fc polypeptide. As another example, in some cases, a TMP comprises a heterodimer, wherein the heterodimer comprises: a) a first polypeptide comprising, in order from N-terminus to C-terminus: i) a cancer-associated peptide; ii) a peptide linker; and iii) a β2M polypeptide; and b) a second polypeptide comprising, in order from N-terminus to C-terminus: i) an MHC class I heavy chain polypeptide; ii) an optional peptide linker; iii) an Ig Fc polypeptide; iv) an optional peptide linker; v) and one or more MODs (where, if the TMP comprises two MODs in tandem, the MODs may be separated by a peptide linker. In some cases, the heterodimer comprises a disulfide bond formed between a Cys residue in the β2M polypeptide and a Cys residue in the MHC class I heavy chain polypeptide. In some cases, the heterodimer comprises a disulfide bond formed between (i) a Cys residue in a Cys-containing linker interposed between the cancer-associated peptide and the β2M polypeptide, and (ii) a Cys residue in the MHC class I heavy chain polypeptide. In some cases, the first polypeptide and the second polypeptide are covalently linked to one another by at least a first and second disulfide bond, where: a) the first disulfide bond formed between (i) a Cys residue in a Cys-containing linker between the cancer-associated peptide and the β2M polypeptide, and (ii) a Cys residue in the MHC class I heavy chain polypeptide; and b) the second disulfide bond is formed between a Cys residue in the β2M polypeptide and a Cys residue in the MHC class I heavy chain polypeptide.

### Single-Chain TMPs

As noted above, in some cases, a TMP is a single-chain polypeptide. The components of the single-chain TMP can be arranged in any of a variety of configurations.

A single-chain TMP comprises: i) a cancer-associated peptide; ii) an optional peptide linker; iii) a β2M polypeptide; iv) an optional peptide linker; v) an MHC class I heavy chain polypeptide; vi) an optional peptide linker; vii) an Ig Fc polypeptide; viii) an optional peptide linker; and ix) one or more immunomodulatory polypeptides (where, if the TMP comprises two MODs in tandem, the MODs may be separated by a peptide linker). In some cases, the TMP comprises a Cys-containing peptide linker between the cancer-associated peptide and the β2M polypeptide, and the TMP comprises an intrachain disulfide bond (i.e., a linker disulfide) between the Cys present in the peptide linker and a Cys in the MHC class I heavy chain polypeptide. In some cases, the single-chain TMP comprises an intrachain disulfide bond (i.e., a body disulfide) formed between a Cys residue in the β2M polypeptide and a Cys residue in the MHC class I heavy chain polypeptide. In some cases, the single-chain TMP comprises both a linker disulfide and one or more body disulfides.

### Interchain and Intrachain Disulfide Bonds

As discussed above, the first and the second polypeptide of a heterodimer of a TMP can be covalently linked to one another by one or more disulfide bonds, e.g., a linker disulfide and/or one or more body disulfides. Where a TMP is a single-chain polypeptide, the single-chain TMP can comprise one or more intrachain disulfide bonds, e.g., a linker disulfide and/or one or more body disulfides.

In some cases, the first and the second polypeptides of a heterodimeric TMP are covalently linked to one another by a single disulfide bond. In some cases, the single disulfide bond is a body disulfide between a Cys in the β2M polypeptide and a Cys in the MHC class I heavy chain polypeptide. In some cases, the Cys in the β2M polypeptide is amino acid 12 of the β2M polypeptide. In some cases, the Cys in the MHC class I heavy chain polypeptide is at amino acid 236. In some cases, the first and the second polypeptides of a heterodimeric TMP are covalently linked to one another by a linker disulfide bond between: i) a Cys in the peptide linker between the cancer-associated peptide and the β2M polypeptide; and ii) a Cys in the MHC class I heavy chain polypeptide. In some cases, the first and the second polypeptides of a heterodimeric TMP are covalently linked to one another by at least a first and second disulfide bond, where the first disulfide bond is a body disulfide and the second disulfide is a linker disulfide In some cases where the TMP comprises a linker disulfide, the peptide linker between the cancer-associated peptide and the β2M polypeptide comprises the sequence CGGGS(GGGGS)n (SEQ ID NO:68), GCGGS(GGGGS)n (SEQ ID NO:69), or GGCGS(GGGGS)n (SEQ ID NO:70), wherein n is an integer from 1-10. In some cases, the second disulfide bond is formed between: i) the Cys in the peptide linker between the cancer-associated peptide and the β2M polypeptide; and ii) a Cys at amino acid 84 of the MHC class I heavy chain polypeptide. In some cases, the second disulfide bond is formed between the Cys in the peptide linker and a Cys at any one of amino acids 135-143 of the MHC class I heavy chain polypeptide. In some cases, the second disulfide bond is formed between: i) the Cys in the peptide linker between the cancer-associated peptide and the β2M polypeptide; and ii) a Cys at amino acid 139 of the MHC class I heavy chain polypeptide.

In some cases, a single-chain TMP includes a single intrachain disulfide bond. In some cases, the single disulfide bond is between a Cys in the β2M polypeptide and a Cys in the MHC class I heavy chain polypeptide. In some cases, the Cys in the β2M polypeptide is amino acid 12 of the β2M polypeptide. In some cases, the Cys in the MHC class I heavy chain polypeptide is at amino acid 236. In some cases, a single-chain TMP includes 2 intrachain disulfide bonds. In some cases, the second disulfide bond is between: i) a Cys in the peptide linker between the cancer-associated peptide and the β2M polypeptide; and ii) a Cys in the MHC class I heavy chain polypeptide. In some cases, the peptide linker between the cancer-associated peptide and the β2M polypeptide comprises the sequence CGGGS(GGGGS)n (SEQ ID NO:68), GCGGS(GGGGS)n (SEQ ID NO:69), or GGCGS(GGGGS)n (SEQ ID NO:70), wherein n is an integer from 1-10. In some cases, the second disulfide bond is formed between: i) the Cys in the peptide linker between the cancer-associated peptide and the β2M polypeptide; and ii) a Cys at amino acid 84 of the MHC class I heavy chain polypeptide. In some cases, the second disulfide bond is formed between the Cys in the peptide linker and a Cys at any one of amino acids 135-143 of the MHC class I heavy chain polypeptide. In some cases, the second disulfide bond is formed between: i) the Cys in the peptide linker between the cancer-associated peptide and the β2M polypeptide; and ii) a Cys at amino acid 139 of the MHC class I heavy chain polypeptide.

### Exemplary TMPs

In some cases, a TMP comprises a heterodimer comprising: a) a first polypeptide comprising, in order from N-terminus to C-terminus: i) a cancer-associated peptide; ii) a peptide linker; and iii) a β2M polypeptide; and b) a second polypeptide comprising, in order from N-terminus to C-terminus: i) a first immunomodulatory polypeptide; ii) a peptide linker; iii) a second immunomodulatory polypeptide; iv) an MHC class I heavy chain polypeptide; v) a peptide linker; and vi) an Ig Fc polypeptide.

In some cases, the cancer-associated peptide is an HPV16 E7 peptide. In some cases, the cancer-associated peptide has the amino acid sequence: YMLDLQPETT (SEQ ID NO:20). In some cases, the linker between the cancer-associated peptide and the β2M polypeptide comprises the amino acid sequence (GGGGS)3 (SEQ ID NO:71). In some cases, the cancer-associated peptide is a WT-1 peptide.

In some cases, the MHC class I heavy chain polypeptide comprises an amino acid sequence having at least 90%, at least 95%, at least 98%, at least 99%, or 100%, amino acid sequence identity to the following amino acid sequence: GSHSMRYFFTSVSRPGRGEPRFIAVGYVDDTQFVRFDSDAASQRMEPRAPWIEQEGPEYWDGET RKVKAHSQTHRVDLGTLRGAYNQSEAGSHTVQRMYGCDVGSDWRFLRGYHQYAYDGKDYIA LKEDLRSWTAADMAAQTTKHKWEAAHVAEQLRAYLEGTCVEWLRRYLENGKETLQRTDAPK THMTHHAVSDHEATLRCWALSFYPAEITLTWQRDGEDQTQDTELVETRPCGDGTFQKWAAVV VPSGQEQRYTCHVQHEGLPKPLTLRWE (SEQ ID NO:4; FIG. 7D). In some cases, amino acid 84 is an Ala and amino acid 236 is a Cys.

In some cases, the β2M polypeptide comprises an amino acid sequence having at least 90%, at least 95%, at least 98%, at least 99%, or 100%, amino acid sequence identity to the following amino acid sequence: IQRTPKIQVY SCHPAENGKS NFLNCYVSGF HPSDIEVDLLKNGERIEKVE HSDLSFSKDW SFYLLYYTEF TPTEKDEYAC RVNHVTLSQP KIVKWDRDM (SEQ ID NO:2; FIG. 7B). In some cases, amino acid 12 is a Cys.

In some cases, the TMP comprises two copies of a variant IL-2 polypeptide comprising an amino acid sequence having at least 90%, at least 95%, at least 98%, at least 99%, or 100%, amino acid sequence identity to the following amino acid sequence: APTSSSTKKT QLQLEX₁LLLD LQMILNGINN YKNPKLTRML TX₂KFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:21), where X₁ is other than His, e.g., Ala, Thr, Glu or Asp; and X₂ is other than Phe, e.g., Ala. In some cases, amino acid 16 is an Ala and amino acid 42 is an Ala.

In some cases, e.g., where the Ig Fc is on the C-terminus of the second polypeptide of a heterodimeric TMP, the Ig Fc polypeptide comprises an amino acid sequence having at least 90%, at least 95%, at least 98%, at least 99%, or 100%, amino acid sequence identity to the following amino acid sequence: DKTHTCPPCPAPEAAGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKAKGQPREPQ VYTLPPSREEMTKNQVSLTCLVKGFYPSDIAVEWESNGQPENNYKTTPPVLDSDGSFFLYSKLT VDKSRWQQGNVFSCSVMHEALHNHYTQKSLSLSPG (SEQ ID NO:9; FIG. 7I). In some cases, amino acids 14 and 15 are both Ala, and the Ig Fc polypeptide optionally does not comprise a Lys at the C-terminus.

In some cases, including cases where the Ig Fc is on the C-terminus of the second polypeptide of a heterodimeric TMP, the Ig Fc polypeptide comprises an amino acid sequence having at least 90%, at least 95%, at least 98%, at least 99%, or 100%, amino acid sequence identity to the following amino acid sequence: DKTHTCPPCPAPEAAGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKAKGQPREPQ VYTLPPSREEMTKNQVSLTCLVKGFYPSDIAVEWESNGQPENNYKTTPPVLDSDGSFFLYSKLT VDKSRWQQGNVFSCSVMHEALHNHYTQKSLSLSPGK (SEQ ID NO:10; FIG. 7J). In some cases, amino acids 14 and 15 are both Ala, and the Ig Fc does comprise a Lys at the C-terminus.

In some cases, a TMP is a heterodimer comprising: a) a first polypeptide comprising: i) a cancer-associated peptide having the amino acid sequence YMLDLQPETT (SEQ ID NO:20); ii) a peptide linker having the amino acid sequence (GGGGS)n (SEQ ID NO:72), where n is an integer from 1 to 10 (e.g., where n is 2, 3, or 4); iii) a β2M polypeptide comprising an amino acid sequence having at least 90%, at least 95%, at least 98%, at least 99%, or 100%, amino acid sequence identity to the following amino acid sequence: IQRTPKIQVYSCHPAENGKSNFLNCYVSGFHPSDIEVDLLKNGERIEKVEHSDLSFSKDWSFYLL YYTEFTPTEKDEYACRVNHVTLSQPKIVKWDRDM (SEQ ID NO:2); and b) a second polypeptide comprising, in order from N-terminus to C-terminus:
i) a first variant IL-2 polypeptide comprising an amino acid sequence having at least 90%, at least 95%, at least 98%, at least 99%, or 100%, amino acid sequence identity to the following amino acid sequence: APTSSSTKKT QLQLEALLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:22);
ii) a peptide linker having the amino acid sequence (GGGGS)n (SEQ ID NO:72), where n is an integer from 1 to 10 (e.g., where n is 2, 3, or 4);
iii) a second variant IL-2 polypeptide comprising an amino acid sequence having at least 90%, at least 95%, at least 98%, at least 99%, or 100%, amino acid sequence identity to the following amino acid sequence: APTSSSTKKT QLQLEALLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:22);
iv) a peptide linker having the amino acid sequence (GGGGS)n (SEQ ID NO:72), where n is an integer from 1 to 10 (e.g., where n is 2, 3, or 4);
v) an MHC class I heavy chain polypeptide comprising an amino acid sequence having at least 90%, at least 95%, at least 98%, at least 99%, or 100%, amino acid sequence identity to the following amino acid sequence:

(SEQ ID NO: 4; FIG. 7D)
GSHSMRYFFTSVSRPGRGEPRFIAVGYVDDTQFVRFDSDAASQRMEPRA
PWIEQEGPEYWDGETRKVKAHSQTHRVDLGTLRGAYNQSEAGSHTVQRM
YGCDVGSDWRFLRGYHQYAYDGKDYIALKEDLRSWTAADMAAQTTKHKW
EAAHVAEQLRAYLEGTCVEWLRRYLENGKETLQRTDAPKTHMTHHAVSD
HEATLRCWALSFYPAEITLTWQRDGEDQTQDTELVETRPCGDGTFQKWA
AVVVPSGQEQRYTCHVQHEGLPKPLTLRWE;

vi) a peptide linker, e.g., a peptide comprising the amino acid sequence AAAGG (SEQ ID NO: 73);
v) an Ig Fc polypeptide comprising an amino acid sequence having at least 90%, at least 95%, at least 98%, at least 99%, or 100%, amino acid sequence identity to the following amino acid sequence: DKTHTCPPCPAPEAAGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKAKGQPREPQ VYTLPPSREEMTKNQVSLTCLVKGFYPSDIAVEWESNGQPENNYKTTPPVLDSDGSFFLYSKLT VDKSRWQQGNVFSCSVMHEALHNHYTQKSLSLSPG (SEQ ID NO:9; FIG. 7I), wherein the Ig Fc optionally includes a Lys at the C terminus.

In some cases, a TMP is a heterodimer comprising: a) a first polypeptide comprising the amino acid sequence YMLDLQPETTGGGGSGGGGSGGGGSIQRTPKIQVYSCHPAENGKSNFLNCYVSGFHPSDIEVD LLKNGERIEKVEHSDLSFSKDWSFYLLYYTEFTPTEKDEYACRVNHVTLSQPKIVKWDRDM (SEQ ID NO:19; FIG. 7S); and b) a second polypeptide comprising, in order from N-terminus to C-terminus:
i) a first variant IL-2 polypeptide comprising the amino acid sequence: APTSSSTKKT QLQLEALLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:22);
ii) a peptide linker comprising the amino acid sequence (GGGGS)4 (SEQ ID NO:74);
iii) a second variant IL-2 polypeptide comprising the amino acid sequence: APTSSSTKKT QLQLEALLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:22);
iv) a peptide linker comprising the amino acid sequence (GGGGS)4 (SEQ ID NO:74);
v) an MHC class I heavy chain polypeptide comprising the amino acid sequence:

(SEQ ID NO: 4; FIG. 7D)
GSHSMRYFFTSVSRPGRGEPRFIAVGYVDDTQFVRFDSDAASQRMEPRA
PWIEQEGPEYWDGETRKVKAHSQTHRVDLGTLRGAYNQSEAGSHTVQRM
YGCDVGSDWRFLRGYHQYAYDGKDYIALKEDLRSWTAADMAAQTTKHKW
EAAHVAEQLRAYLEGTCVEWLRRYLENGKETLQRTDAPKTHMTHHAVSD
HEATLRCWALSFYPAEITLTWQRDGEDQTQDTELVETRPCGDGTFQKWA
AVVVPSGQEQRYTCHVQHEGLPKPLTLRWE;

vi) a peptide linker comprising the amino acid sequence AAAGG (SEQ ID NO:73);
v) an Ig Fc polypeptide comprising the amino acid sequence:

(SEQ ID NO: 9; FIG. 7I)
DKTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSR
WQQGNVFSCSVMHEALHNHYTQKSLSLSPG.

In some cases, a TMP is a heterodimer comprising: a) a first polypeptide comprising the amino acid sequence YMLDLQPETTGGGGSGGGGSGGGGSIQRTPKIQVYSCHPAENGKSNFLNCYVSGFHPSDIEVD LLKNGERIEKVEHSDLSFSKDWSFYLLYYTEFTPTEKDEYACRVNHVTLSQPKIVKWDRDM (SEQ ID NO:19; FIG. 7S); and b) a second polypeptide comprising the amino acid sequence depicted in FIG. 7Q (SEQ ID NO:17).

In some cases, a TMP is a heterodimer comprising: a) a first polypeptide comprising the amino acid sequence YMLDLQPETTGGGGSGGGGSGGGGSIQRTPKIQVYSCHPAENGKSNFLNCYVSGFHPSDIEVD LLKNGERIEKVEHSDLSFSKDWSFYLLYYTEFTPTEKDEYACRVNHVTLSQPKIVKWDRDM (SEQ ID NO:19; FIG. 7S); and b) a second polypeptide comprising the amino acid sequence depicted in FIG. 7R (SEQ ID NO:18).

### Multimers of a TMP

As discussed above, a TMP can be a dimer of two TMPs. That is, the present disclosure provides a TMP comprising a dimer of two TMPs. The covalent linkage of the dimer can be one or more (typically two) disulfide bonds between an Ig Fc polypeptide in the first TMP and an Ig Fc polypeptide in the second TMP. When the TMP comprises an Ig Fc polypeptide, the TMP typically will self-assemble into a dimer by spontaneously forming one or more (typically two) disulfide bonds with the Ig Fc polypeptide of another TMP. Thus, e.g., the Ig Fc polypeptides in the first TMP and the second TMP can be linked to one another by one or more (e.g., two) disulfide bonds. In many cases, the two TMPs will be identical to one another in amino acid sequence and comprise Ig Fc polypeptides that spontaneously form one or more (typically two) disulfide bonds, thereby forming a TMP that is a homodimer. Alternatively, the TMP dimer may be heterodimers of the two TMPs. In such case, the Ig Fc polypeptides of each TMP can comprise interspecific dimerization sequences, e.g., “Knob-in-Hole” sequences that permit two different TMPs to selectively dimerize. Such interspecific dimerization sequences are disclosed in WO 2022/197970 (Cue Biopharma, Inc.), the disclosure of which as it pertains to interspecific dimerization sequences (including but not limited to [00212]-[00213]) is expressly incorporated herein by reference. Interspecific dimerization sequences also may be employed to enable TMPs to be linked to non-TMP molecules that can provide additional functionality to the TMP. For example, a TMP could be linked to a molecule that comprise polypeptides (e.g., antibodies or binding fragments thereof such as scFvs) that bind to cancer-associated antigens, thereby enabling the TMP to localize to tissues comprising the cancer-associated antigen.

The TMP dimers include homodimers and heterodimers of heterodimeric TMPs. See, e.g., See, e.g., WO 2018/119114 and WO 2020/132136 (both to Cue Biopharma, Inc.), the disclosures of which as they pertain to such TMP dimers (referred to therein as “T-cell multimeric polypeptides”) is expressly incorporated by reference.

The TMP dimers include homodimers and heterodimers of single-chain TMPs. See, e.g., WO 2022/197970 (Cue Biopharma, Inc.), the disclosure of which as it pertains to such TMP dimers (including but not limited to [00208]-[00213]) is expressly incorporated herein by reference.

As discussed above, the TMP dimers also include heterodimers and homodimers of TMPs that are is a multimeric antigen-presenting polypeptides (MAPPs) as described in WO 2021/242935 (Cue Biopharma, Inc.) or WO 2022/099157 (Cue Biopharma, Inc.), the disclosures of which as they pertain to dimers of MAPPS are expressly incorporated by reference.

It should be understood that references to TMPs in the sections that follow expressly include TMP dimers, i.e., both homodimers and heterodimers.

### PD-(L)1 CPIs

PD-(L)1 CPIs include antibodies that target, i.e., specifically bind to, PD-1 or PD-L1. Exemplary anti-PD-1 antibodies include nivolumab (OPDIVO®), pembrolizumab (KEYTRUDA®), cemiplimab (LIBTAYO®), dostarlimab (JEMPERLI®), retifanlimab (ZYNYX®), toripalimab (LOQTORZ®) and tislelizumab (TEVIMBRA®). Exemplary anti-PD-L1 antibodies include durvalumab (IMFINZI®), atezolizumab (TECENTRIQ®), and avelumab (BAVENCIO®).

### Formulations, Dosages, and Routes of Administration of the TMP

A composition can comprise, in addition to a TMP, one or more pharmaceutically acceptable additives, a variety of which are known in the art and need not be discussed in detail herein. See, for example, the ninth (or latest) edition of Sheskey et al., “Handbook of Pharmaceutical Excipients” (2020), and/or the 23^{rd} (or latest) edition of “Remington: The Science and Practice of Pharmacy”, 23rd Ed. (2020).

Where a TMP is administered as an injectable (e.g. subcutaneously, intraperitoneally, intramuscularly, and/or intravenously) directly into a tissue, a formulation can be provided as a ready-to-use dosage form that may be directly injected or infused into the patient or admixed with a saline solution for infusion, or possibly as a non-aqueous form (e.g., a reconstitutable storage-stable powder) or aqueous form, such as liquid composed of pharmaceutically acceptable carriers and excipients. Formulations may also be provided so as to enhance serum half-life of the TMP following administration. For example, the TMP may be provided in a liposome formulation, prepared as a colloid, or other conventional techniques for extending serum half-life. The preparations may also be provided in controlled release or slow-release forms.

The concentration of a TMP in a liquid composition formulation can vary widely (e.g., from less than about 0.1%, usually at or at least about 2% to as much as 20% to 50% or more by weight). Included within this range is a concentration of from about 5 to about 15 mg/mL, from about 8 to about 12 mg/mL, from about 9 to about 11 mg/mL, including about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL and about 15 mg/mL. The concentration may depend on numerous factors, including the stability of the TMP in the liquid composition.

In some cases, a TMP is present in a liquid composition. In some cases, a composition comprises: a) a TMP; and b) saline (e.g., 0.9% NaCl). In some cases, the composition is sterile and suitable for administration to a human subject.

In some cases, a recombinant expression vector comprising one or more nucleic acids encoding a TMP (or a cell, e.g., a B cell or other blood cell such as a red blood cell comprising such nucleic acids or recombinant expression vector) may be administered to induce in vivo expression of the TMP instead of directly administering the TMP itself to a patient.

A suitable dose of a TMP can be determined by an attending physician or other qualified medical personnel, based on various clinical factors. As is well known in the medical arts, dosages for any one patient depend upon many factors, including the patient's size, body surface area, age, the particular TMP or nucleic acid to be administered (including the number and type of MOD(s) that are part of the TMP), sex of the patient, time, and route of administration, general health, and other drugs being administered concurrently. A TMP may be administered in amounts between 0.1 mg/kg body weight and 20 mg/kg body weight per dose, e.g. between 0.1 mg/kg body weight to 10 mg/kg body weight, e.g., from 0.5 mg/kg to 1 mg/kg, from 0.5 mg/kg body weight to 5 mg/kg body weight, from 1 mg/kg body weight to 5 mg/kg body weight; from 5 mg/kg body weight to 10 mg/kg body weight; from 10 mg/kg body weight to 15 mg/kg body weight; from 15 mg/kg body weight to 20 mg/kg body weight, however, doses above this exemplary range are envisioned, especially considering the aforementioned factors. If the regimen is a continuous infusion, it can also be in the range of 1 μg to 10 mg per kilogram of body weight per minute.

Alternatively, a patient may simply be administered a flat amount of a TMP, e.g., in an amount of from 200 mg to 600 mg, including from 250 mg to 450 mg, from 250 mg to 350 mg, from 450 mg to 600 mg, including specific amounts of 250 mg, 300 mg, 350 mg, 400 mg, 500 mg or 600 mg.

When administered in combination with the PD-(L)1 CPI, in order to reduce or minimize the amount of unwanted side effects (e.g., adverse events having a severity greater than 2), it may be preferable to employ a dose of the TMP that is lower than the dose of the TMP that would be administered if the TMP were administered as a monotherapy. For example, a dose of TMP between 0.5 mg/kg and 1 mg/kg may be preferred in some cases.

Where the TMP includes one or more variant IL-2 MODs, or where a dimerized TMP comprises 2 or more (e.g., 4) variant IL-2 MODs such as those discussed above that comprise H16 and F42 substitutions, exemplary amounts of TMP include from 1 mg/kg body weight to 5 mg/kg body weight, from 5 mg/kg body weight to 10 mg/kg body weight, from about 1 mg/kg body weight to about 5 mg/kg body weight, and from about 5 mg/kg body weight to about 10 mg/kg body weight. When the TMP comprises four of such variant IL-2 MODs, exemplary amounts of TMP include 1 mg/kg, 2 mg/kg, 3 mg/kg, 4 mg/kg or 5 mg/kg, e.g., 2 mg/kg, 3 mg/kg or 4 mg/kg. As noted above, it may be preferred to administer a lower dose, e.g., from 0.5 mg/kg to 1 mg/kg when co-administering the TMP with a PD-(L)1 CPI.

Persons of ordinary skill in the art can easily estimate repetition rates for dosing based on measured residence times and concentrations of the administered agent in bodily fluids or tissues. Following successful treatment, it may be desirable to have the patient undergo maintenance therapy to prevent the recurrence of the disease state, wherein a TMP or dimerized TMP is administered in maintenance doses, ranging from about 1 mg/kg body weight to about 5 mg/kg body weight (e.g., 1-5 mg/kg), from about 5 mg/kg body weight to about 10 mg/kg body weight (e.g., 5-10 mg/kg), from about 10 mg/kg body weight to about 15 mg/kg body weight (e.g., 10-15 mg/kg), from about 15 mg/kg body weight to about 20 mg/kg body weight (e.g., 15-20 mg/kg), or amounts exceeding 20 mg/kg of body weight.

Those of skill will readily appreciate that dose levels can vary as a function of the specific TMP, the severity of the symptoms and the susceptibility of the subject to side effects. Preferred dosages for a given compound are readily determinable by those of skill in the art by a variety of means.

In some cases, multiple doses of a TMP, a nucleic acid, or a recombinant expression vector (or a cell, e.g., a B cell or other blood cell such as a red blood cell comprising a nucleic acid or recombinant expression vector) are administered. The frequency of administration of a TMP, a nucleic acid, or a recombinant expression vector can vary depending on any of a variety of factors, e.g., severity of unwanted side effects, etc. For example, in some cases, a TMP, a nucleic acid, or a recombinant expression vector (or a cell, e.g., a B cell or a blood cell such as a red blood cell comprising a nucleic acid or recombinant expression vector) is administered once per month, twice per month, three times per month, every other week (qow), once per week (qw), once every two weeks, once every three weeks, or once every four weeks. It also is possible to administer a TMP more often than once per week, i.e., multiple times per week. Where the TMP is administered intravenously, administration once every week, once every two weeks, once every three weeks or once every four weeks or once every month may be commonly employed at the beginning of treatment. In order to minimize the disruption to the life of the patient, it may be desirable to administer the TMP at the same time as the PD-(L)1 CPI, e.g., once every three weeks in the case of pembrolizumab. It also may be that the frequency of administration for the TMP and PD-(L)1 CPI are different. For example, whereas 200 mg of pembrolizumab may be administered every three weeks, administration of 400 mg every six weeks is also approved for most or all indications where pembrolizumab is approved. Hence, a TMP having a dosing frequency of once every three weeks might be administered with 200 mg of pembrolizumab at the same time every three weeks or receive 400 mg of the pembrolizumab only once every six weeks, i.e., half as often as the TMP.

The duration of administration of a TMP, a nucleic acid, or a recombinant expression vector (or a cell, e.g., a B cell or a blood cell such as a red blood cell comprising a nucleic acid or recombinant expression vector), e.g., the period of time over which a TMP, a nucleic acid, or a recombinant expression vector is administered, can vary, depending on any of a variety of factors, e.g., patient response, etc. For example, a TMP, a nucleic acid, or a recombinant expression vector (or a cell, e.g., a B cell or a blood cell such as a red blood cell comprising a nucleic acid or recombinant expression vector) can be administered over a period of time ranging from weeks to months and continued until the patient is determined to have progressive disease under standard RECIST criteria, e.g., RECIST 1.1. The length of time may depend on whether the TMP is administered in a neoadjuvant setting, in which case it may be administered only once or a few times, e.g., two, three or four times, for only a week or a few weeks before a main treatment such as surgery. In an adjuvant setting or a setting in which the patient is being treated for a cancer that has newly appeared or is recurrent or metastatic, the treatment duration may continue indefinitely or until the patient exhibits progressive disease as determined by standard RECIST criteria such as RECIST 1.1.

### Cancers Suitable for Treatment

Cancers that can be treated with a method of the present disclosure include cancers associated with HPV16 infections. Cancers that can be treated with a method include, e.g., newly diagnosed or recurrent and/or metastatic: HPV16+ head and neck cancers such as squamous cell carcinoma, oropharyngeal cancer, nasopharyngeal cancer, hypopharyngeal cancer, and laryngeal cancer; anal cancer; penile cancer; vulvar cancer; vaginal cancer; and cervical cancer.

### Methods of Treatment

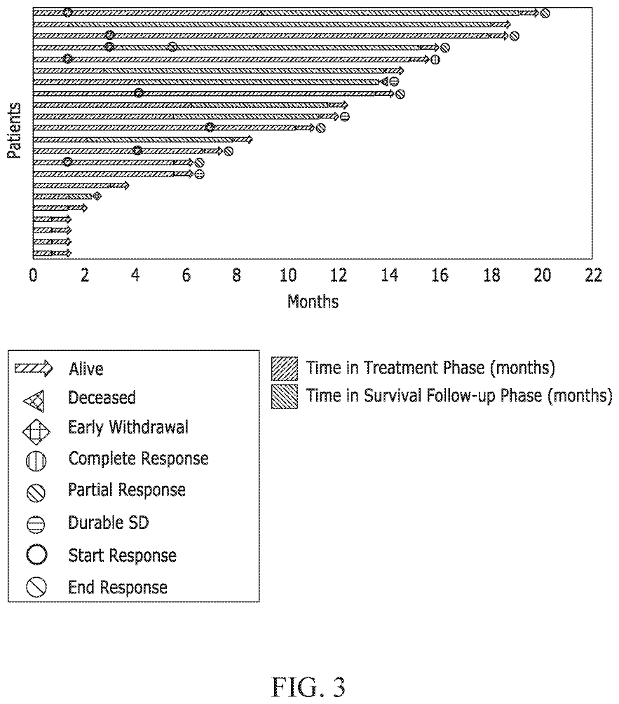

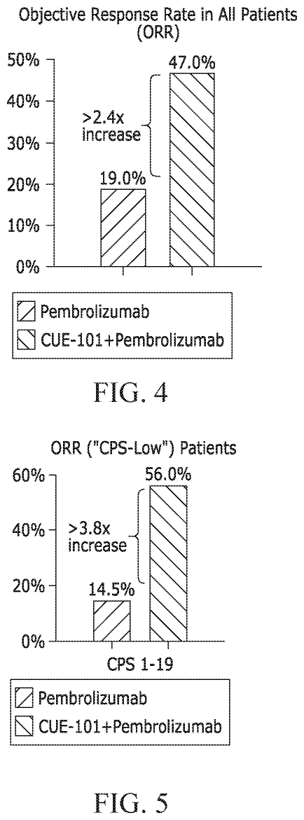

As discussed below in Example 1, based on the clinical trial KEYNOTE-A78 involving seventeen evaluable patients to date, the ORR for patients who have CPS≥1, as determined by RECIST 1.1 criteria, currently is significantly greater for patients receiving the combination of a TMP (CUE-101) and the anti-PD-1 CPI pembrolizumab (47%) than for pembrolizumab alone (19%). See FIG. 3. Further, the increase in ORR for patients who have low CPS, i.e., from 1-19, is currently even greater for patients receiving the combination of the TMP and the anti-PD-1 CPI (56%) than for the anti-PD-1 CPI alone (14.5%). See FIG. 4. And patients having a PD-L1 CPS≥20 who received the combination of the TMP and the anti-PD-1 CPI exhibited a significantly greater ORR (38%) than those who received the anti-PD-1 checkpoint inhibitor alone (23%). See FIG. 5.

Accordingly, patients who were previously treated with a PD-(L)1 CPI such as pembrolizumab, nivolumab, cemiplimab or durvalumab, either alone or in combination with one or more therapeutic agents (e.g., one or more chemotherapeutic agents), not including a TMP of this disclosure, and were determined to have progressive disease based on RECIST 1.1 criteria may benefit from receiving a combination treatment of an effective amount of a TMP of this disclosure, e.g., CUE-101 (described below), in combination with a PD-(L)1 CPI such as pembrolizumab, nivolumab, cemiplimab or durvalumab. Such patients may have a PD-L1 CPS score that is 0, more than 0 but less than 1, from 1 to 19, from 1-20, from 0 to 100, from more than 0 to 100, from 1 to 100, from 20 to 25, from 25 to 50, from 50 to 75, or greater than 75.

Patients who have a PD-L1 CPS of 0 or more than 0 to less than 1, whether newly diagnosed, or having been previously diagnosed with cancer, may also benefit from receiving the combination of a TMP and PD-(L)1 CPI. Such patients may include individuals who (i) had no prior therapies following an initial diagnosis of HPV16+ cancer, (ii) had at least one prior therapy following an initial diagnosis of HPV16+ cancer and have been determined to have progressive disease, (iii) are receiving the combination as a neoadjuvant therapy for an HPV16+ cancer, (iv) are receiving the combination as a adjuvant therapy for an HPV16+ cancer, (v) are receiving the combination as a first line treatment following a diagnosis of recurrent and/or metastatic HPV16+ cancer, or (vi) are receiving the combination as a second line or beyond therapy following a diagnosis of recurrent and/or metastatic HPV16+ cancer and treatment with a prior therapy following the diagnosis of recurrent and/or metastatic HPV16+ cancer and have been diagnosed with progressive disease following such prior treatment. Such patients also may include individuals who, due to having a PD-L1 CPS below 1, might not qualify for treatment with some PD-(L)1 CPIs such as pembrolizumab (either alone or in combination with one or more therapeutic agents such as chemotherapeutic agents).

While not wishing to be bound by a particular theory, it is believed that the increase in the number and activation of tumor-specific T cells brought about by the TMP may cause an upregulation in the expression of checkpoints such as PD-1 on T cells and/or the expression of checkpoints such as PD-L1 on cancer cells such that the presence of the PD-(L)1 CPI can be beneficial for the effectiveness of treating the cancer when such upregulated expression occurs.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below.
1. A method of treating an HPV16-associated cancer in an individual, the method comprising administering to the individual a combination of (i) a PD-(L)1 CPI, and (ii) a T cell modulating protein (TMP), wherein the TMP comprises:
i) an HPV16 cancer-associated peptide, wherein the HPV16 cancer-associated peptide is a peptide having a length of from 8 amino acids to 14 amino acids;
ii) a β2-microglobulin (β2M) polypeptide;
iii) one or more immunomodulatory polypeptides;
iv) a class I major histocompatibility complex (MHC) heavy chain polypeptide; and
v) a scaffold component, optionally wherein the scaffold component is an immunoglobulin (Ig) Fc polypeptide,
wherein the TMP may comprise one or more independently selected peptide linkers between any two of the components, and wherein when the TMP comprises more than one immunomodulatory polypeptide, the immunomodulatory polypeptides may be connected by one or more independently selected peptide linkers,
wherein the individual was previously determined to have progressive disease based on RECIST 1.1 criteria following (i) monotherapy with a PD-(L)1 CPI, or (ii) combination therapy comprising treatment with a PD-(L)1 CPI and one or more therapeutic agents (optionally one or more chemotherapeutic agents) other than the TMP, and
wherein the PD-(L)1 CPI and the TMP are administered at the same time or at different times.
2. The method of aspect 1, wherein the cancer is associated with a PD-L1 Combined Positive Score (PD-L1 CPS) that is 20 or less, or 19 or less.
3. The method of aspect 1, wherein the cancer is associated with a PD-L1 CPS that is 20 or greater, or 21 or greater.
4. The method of aspect 1, wherein the cancer is associated with a PD-L1 CPS that is either 0 or more than 0 but less than 1.
5. A method of treating an HPV16-associated cancer in an individual, the method comprising administering to the individual a combination of (i) a PD-(L)1 CPI, and (ii) a T cell modulating protein (TMP), wherein the TMP comprises:
i) an HPV16 cancer-associated peptide, wherein the HPV16 cancer-associated peptide is a peptide having a length of from 8 amino acids to 14 amino acids;
ii) a β2-microglobulin (β2M) polypeptide;
iii) one or more immunomodulatory polypeptides;
iv) a class I major histocompatibility complex (MHC) heavy chain polypeptide; and
v) a scaffold component, optionally wherein the scaffold component is an immunoglobulin (Ig) Fc polypeptide,
wherein the TMP comprises one or more independently selected peptide linkers between any two of the components, and wherein when the TMP comprises more than one immunomodulatory polypeptide, the immunomodulatory polypeptides may be connected by one or more independently selected peptide linkers,
wherein the PD-(L)1 CPI and the protein are administered at the same time or at different times, and
wherein the cancer is associated with a PD-L1 Combined Positive Score (PD-L1 CPS) that is either 0 or more than 0 but less than 1.
6. The method of aspect 5, wherein
the combination is administered to an individual who (i) had no prior therapies following an initial diagnosis of HPV16+ cancer, (ii) had at least one prior therapy following an initial diagnosis of HPV16+ cancer and was determined to have progressive disease, (iii) is receiving the combination as a neoadjuvant therapy for an HPV16+ cancer, (iv) is receiving the combination as a adjuvant therapy for an HPV16+ cancer, (v) is receiving the combination as a first line treatment following a diagnosis of recurrent and/or metastatic HPV16+ cancer, or (vi) is receiving the combination as a second line or beyond therapy following a diagnosis of recurrent and/or metastatic HPV16+ cancer and the patient has been diagnosed with progressive disease, and
wherein optionally the individual previously was not qualified for treatment with a PD-(L)1 CPI, either alone or in combination with one or more therapeutic agents, e.g., one or more chemotherapeutic agents.
7. The method of any one of aspects 1-5, wherein the PD-L1 CPS is determined by an Approved Laboratory using:
(i) a PD-L1 IHC 22C3 pharmDx assay or an assay that employs a monoclonal mouse anti-PD-L1 antibody, clone 22C3;
(ii) a PD-L1 IHC 28-8 pharmDx assay or an assay that employs a monoclonal mouse anti-PD-L1 antibody, clone 28-8; or
(iii) an assay other than (i) or (ii).
8. The method of any one of aspects 1-7, wherein the TMP comprises a heterodimer, wherein the heterodimer comprises:
   a) a first polypeptide comprising:
i) the HPV16 cancer-associated peptide; and
ii) the β2-microglobulin (β2M) polypeptide; and
   b) a second polypeptide comprising:
i) the one or more immunomodulatory polypeptides;
ii) the MHC class I heavy chain polypeptide; and
iii) an immunoglobulin (Ig) Fc polypeptide.
9. The method of aspect 8, wherein:
   a1) the first polypeptide comprises, in order from N-terminus to C-terminus:
i) the HPV16 cancer-associated peptide;
ii) a peptide linker; and
iii) the β2M polypeptide; and
   b1) the second polypeptide comprises, in order from N-terminus to C-terminus:
i) the one or more immunomodulatory polypeptides;
ii) an optional linker;
iii) the MHC class I heavy chain polypeptide;
iv) an optional linker; and
v) the Ig Fc polypeptide, or
   a2) the first polypeptide comprises, in order from N-terminus to C-terminus:
i) the HPV16 cancer-associated peptide;
ii) a peptide linker; and
iii) the β2M polypeptide; and
   b2) the second polypeptide comprises, in order from N-terminus to C-terminus:
i) the MHC class I heavy chain polypeptide;
ii) an optional linker;
iii) the Ig Fc polypeptide;
iv) an optional linker; and
v) the one or more immunomodulatory polypeptides,
wherein when the TMP comprises more than one immunomodulatory polypeptide, the TMP may comprise one or more linkers between the immunomodulatory polypeptides.
10. The method of aspect 8 or 9, wherein the heterodimer comprises a disulfide bond formed between a Cys residue in the β2M polypeptide and a Cys residue in the MHC class I heavy chain polypeptide.
11. The method of any one of aspects 8-10, wherein the heterodimer comprises a disulfide bond formed between (i) a Cys residue in a Cys-containing linker interposed between the HPV16 cancer-associated peptide and the β2M polypeptide, and (ii) a Cys residue in the MHC class I heavy chain polypeptide.
12. The method of aspect 8 or 9, wherein the first polypeptide and the second polypeptide are covalently linked to one another by at least a first and second disulfide bond,
wherein the first disulfide bond formed between (i) a Cys residue in a Cys-containing linker between the HPV16 cancer-associated peptide and the β2M polypeptide, and (ii) a Cys residue in the MHC class I heavy chain polypeptide; and
wherein the second disulfide bond is formed between a Cys residue in the β2M polypeptide and a Cys residue in the MHC class I heavy chain polypeptide.
13. The method of any one of aspects 8-12, wherein the one or more immunomodulatory polypeptides are selected from the group of a cytokine, a 4-1BBL polypeptide, an ICOS-L polypeptide, an OX-40L polypeptide, a CD80 polypeptide, a CD86 polypeptide, a CD40 polypeptide, a CD70 polypeptide, and combinations thereof.
14. The method of any one of aspects 8-13, wherein at least one of the one or more immunomodulatory polypeptides is an IL-2 polypeptide.
15. The method of aspect 14, wherein at least one of the one or more immunomodulatory polypeptides comprise a variant IL-2 polypeptide that exhibits reduced affinity to an IL-2 receptor compared to the affinity of a wild-type IL-2 polypeptide for the IL-2 receptor.
16. The method of aspect 15, wherein the variant IL-2 polypeptide has at least 95% amino acid sequence identity to the amino acid sequence set forth in SEQ ID NO:21, wherein amino acid 16 is other than histidine and amino acid 42 is other than phenylalanine.
17. The method of aspect 15, wherein the variant IL-2 polypeptide has at least 98% amino acid sequence identity to the amino acid sequence set forth in SEQ ID NO:21, wherein amino acid 16 is other than histidine and amino acid 42 is other than phenylalanine.
18. The method of aspect 16 or 17, wherein amino acid 16 is Ala, Thr, Asp or Glu.
19. The method of any one of aspects 16-18, wherein amino acid 42 is Ala.
20. The method of aspect 16 or 17, wherein amino acid 16 is Ala and amino acid 42 is Ala.
21. The method of any one of aspects 1-20, wherein the TMP comprises a variant Ig Fc polypeptide comprising one or more amino acid substitutions that reduce or substantially eliminate antibody-dependent cell-mediated cytotoxicity (ADCC) and/or complement-dependent cytotoxicity (CDC).
22. The method of any one of aspects 1-21, wherein the TMP comprises a variant Ig Fc polypeptide comprising an amino acid sequence having at least about 95% amino acid sequence identity to the amino acid sequence set forth in any one of SEQ ID NOs: 7-10 (FIG. 7G-7J).
23. The method of aspect 22, wherein the TMP comprises a variant Ig Fc polypeptide comprising an amino acid sequence having at least about 98% amino acid sequence identity to the amino acid sequence set forth in SEQ ID NO:9 or SEQ ID NO:10, and comprises an Ala at amino acid 14 and an Ala at amino acid 15, based on the amino acid numbering depicted in SEQ ID NO:9.
24. The method of any one of aspects 1-23, wherein the TMP comprises at least two immunomodulatory polypeptides, and wherein at least two of the immunomodulatory polypeptides are the same.
25. The method of any of aspects 15-23, wherein the TMP comprises two variant IL-2 polypeptides in tandem, and wherein the two variant IL-2 polypeptides are optionally connected by a linker.
26. The method of aspect 25, wherein
   a) the first polypeptide of each heterodimer comprises, in order from N-terminus to C-terminus:
i) the HPV16 cancer-associated peptide, wherein the peptide has a length of 8-12 amino acids;
ii) a peptide linker; and
iii) the β2M polypeptide, wherein the β2M polypeptide comprises an amino acid sequence having at least 95% amino acid sequence identity to the amino acid sequence set forth in SEQ ID NO:1 or SEQ ID NO:2;
   b) the second polypeptide of each heterodimer comprises, in order from N-terminus to C-terminus:
i) the first variant IL-2 polypeptide;
ii) a second variant IL-2 polypeptide;
iii) the MHC class I heavy chain polypeptide, wherein the MHC class I heavy chain polypeptide comprises an amino acid sequence having at least 95% amino acid sequence identity to the amino acid sequence set forth in SEQ ID NO:3; and
iv) the Ig Fc polypeptide,
wherein each variant IL-2 polypeptide has at least 95% amino acid sequence identity to the amino acid sequence set forth in SEQ ID NO:21, wherein amino acid 16 is other than histidine and amino acid 42 is other than phenylalanine, and
wherein the second polypeptide comprises an independently selected peptide linker between one or more of:
a) the first immunomodulatory polypeptide and the second immunomodulatory polypeptide;
b) the second immunomodulatory polypeptide and the MHC class I heavy chain polypeptide; and
c) the MHC class I heavy chain polypeptide and the Ig Fc polypeptide.
27. The method of aspect 25, wherein
   a) the first polypeptide of each heterodimer comprises, in order from N-terminus to C-terminus:
i) the HPV16 cancer-associated peptide, wherein the peptide has a length of 8-12 amino acids;
ii) a peptide linker; and
iii) the β2M polypeptide, wherein the β2M polypeptide comprises an amino acid sequence having at least 95% amino acid sequence identity to the amino acid sequence set forth in SEQ ID NO:1 or SEQ ID NO:2;
   b) the second polypeptide of each heterodimer comprises, in order from N-terminus to C-terminus:
i) the MHC class I heavy chain polypeptide, wherein the MHC class I heavy chain polypeptide comprises an amino acid sequence having at least 95% amino acid sequence identity to the amino acid sequence set forth in SEQ ID NO:3;
ii) the Ig Fc polypeptide;
iii) the first variant IL-2 polypeptide; and
iv) a second variant IL-2 polypeptide,
wherein each variant IL-2 polypeptide has at least 95% amino acid sequence identity to the amino acid sequence set forth in SEQ ID NO:21, wherein amino acid 16 is other than histidine and amino acid 42 is other than phenylalanine, and
wherein the second polypeptide comprises an independently selected peptide linker between one or more of:
a) the MHC class I heavy chain polypeptide and the Ig Fc polypeptide
b) the Ig Fc polypeptide the first immunomodulatory polypeptide; and
c) the first immunomodulatory polypeptide and the second immunomodulatory polypeptide.
28. The method of aspect 26 or 27, wherein:
i) the β2M polypeptide comprises a Cys at amino acid 12 based on the amino acid numbering of SEQ ID NO:2; and
ii) the class I MHC polypeptide comprises a Cys at residue 236 based on the amino acid numbering of SEQ ID NO:4, wherein each heterodimer comprises a disulfide bond linking the first polypeptide to the second polypeptide, and wherein the disulfide bond joins the Cys at amino acid 12 of the β2M polypeptide to the Cys at amino acid 236 of the MHC heavy chain polypeptide, and
wherein the Ig Fc polypeptide comprises an amino acid sequence having at least about 95% amino acid sequence identity to the amino acid sequence set forth in SEQ ID NO:9 or SEQ ID NO:10.
29. The method of aspect 28, wherein:
i) the β2M polypeptide comprises the amino acid sequence set forth in SEQ ID NO:2;
ii) the first and second variant IL-2 polypeptides comprise the amino acid sequence set forth in SEQ ID NO:22;
iii) the MHC class I heavy chain polypeptide comprises the amino acid sequence set forth in SEQ ID NO:4; and
iv) the Ig Fc polypeptide comprises an amino acid sequence having at least about 98% amino acid sequence identity to the amino acid sequence set forth in SEQ ID NO:9 or SEQ ID NO: 10, and comprises an Ala at amino acid 14 and an Ala at amino acid 15 based on the amino acid numbering depicted in SEQ ID NO:9, and
   wherein the second polypeptide comprises a peptide linker between:
a) the first and second immunomodulatory polypeptides;
b) the second variant immunomodulatory polypeptide and the MHC class I heavy chain polypeptide; and
c) the MHC class I heavy chain polypeptide and the Ig Fc polypeptide.
30. The method of any one of aspects 1-7, wherein the TMP comprises a single-chain polypeptide, and wherein the single-chain polypeptide comprises from N-terminus to C-terminus:
i) the HPV16 cancer-associated peptide;
ii) an optional peptide linker;
iii) the β2M polypeptide;
iv) an optional peptide linker;
v) the MHC class I heavy chain polypeptide;
vi) an optional peptide linker;
vii) an immunoglobulin (Ig) Fc polypeptide;
viii) an optional peptide linker; and
ix) the one or more immunomodulatory polypeptides.
31. The method of aspect 30, wherein the one or more immunomodulatory polypeptides are selected from the group of a cytokine, a 4-1BBL polypeptide, an ICOS-L polypeptide, an OX-40L polypeptide, a CD80 polypeptide, a CD86 polypeptide, a CD40 polypeptide, a CD70 polypeptide, and combinations thereof.
32. The method of aspect 31, wherein at least one of the one or more immunomodulatory polypeptides is an IL-2 polypeptide.
33. The method of aspect 32, wherein at least one of the one or more immunomodulatory polypeptides comprise a variant IL-2 polypeptide that exhibits reduced affinity to an IL-2 receptor compared to the affinity of a wild-type IL-2 polypeptide for the IL-2 receptor.
34. The method of aspect 33, wherein the variant IL-2 polypeptide variant IL-2 polypeptide has at least 95% amino acid sequence identity to the amino acid sequence set forth in SEQ ID NO:21, wherein amino acid 16 is other than histidine and amino acid 42 is other than phenylalanine.
35. The method of aspect 34, wherein the variant IL-2 polypeptide has at least 98% amino acid sequence identity to the amino acid sequence set forth in SEQ ID NO:21, wherein amino acid 16 is other than histidine and amino acid 42 is other than phenylalanine.
36. The method of aspect 34 or 35, wherein amino acid 16 is Ala, Thr, Asp or Glu.
37. The method of any one of aspects 34-36, wherein amino acid 42 is Ala.
38. The method of aspect 36 or 37, wherein amino acid 16 is Ala and amino acid 42 is Ala.
39. The method of any one of aspects 30-38, wherein the TMP comprises at least two immunomodulatory polypeptides, and wherein at least two of the immunomodulatory polypeptides are the same.
40. The method of any of aspects 33-38, wherein the TMP comprises two variant IL-2 polypeptides in tandem, and wherein the two variant IL-2 polypeptides are optionally connected by a linker.
41. The method of any one of aspects 30-40, wherein
   the β2M polypeptide comprises an amino acid sequence having at least 95% amino acid sequence identity to the amino acid sequence set forth in SEQ ID NO: 1 or SEQ ID NO:2, and
   the MHC class I heavy chain polypeptide comprises an amino acid sequence having at least 95% amino acid sequence identity to the amino acid sequence set forth in SEQ ID NO:3.
42. The method of any one of aspects 30-41, wherein the Ig Fc polypeptide comprises an amino acid sequence having at least about 95% amino acid sequence identity to the amino acid sequence set forth in any one of SEQ ID NOs: 7-10.
43. The method of aspect 42, wherein the Ig Fc polypeptide is a variant Ig Fc polypeptide comprises one or more amino acid substitutions that reduce or substantially eliminate antibody-dependent cell-mediated cytotoxicity (ADCC) and/or complement-dependent cytotoxicity (CDC).
44. The method of aspect 43, wherein the Ig Fc polypeptide comprises an amino acid sequence having at least about 98% amino acid sequence identity to the amino acid sequence set forth in SEQ ID NO:9 or SEQ ID NO: 10, and comprises an Ala at amino acid 14 and an Ala at amino acid 15 based on the amino acid numbering depicted in SEQ ID NO:9.
45. The method of any one of aspects 30-44, wherein the TMP comprises a Cys-containing peptide linker between the cancer-associated peptide and the β2M polypeptide, and wherein the TMP comprises an intrachain disulfide bond between the Cys present in the peptide linker and a Cys in the MHC class I heavy chain polypeptide.
46. The method of aspect 45, wherein the MHC class I heavy chain polypeptide comprises a Cys at any one of amino acids 135-143, based on the numbering of the MHC class I heavy chain polypeptide set forth in SEQ ID NO:6 (depicted in FIG. 7F), and wherein amino acid 84, based on the numbering of the MHC class I heavy chain polypeptide depicted in FIG. 7F, is other than Cys; and wherein the TMP comprises an intrachain disulfide bond between the Cys present in the peptide linker and the Cys at any one of amino acids 135-143 of the MHC class I heavy chain polypeptide.
47. The method of aspect 45, wherein the MHC class I heavy chain polypeptide comprises a Cys at amino acid 138, 139, or 140 based on the numbering of the MHC class I heavy chain polypeptide set forth in SEQ ID NO:6 (depicted in FIG. 7F).
48. The method of aspect 45, wherein the MHC class I heavy chain polypeptide comprises a Cys at amino acid 139 based on the numbering of the MHC class I heavy chain polypeptide set forth in SEQ ID NO:6 (depicted in FIG. 7F).
49. The method of aspect 45, wherein the TMP comprises an intrachain disulfide bond between the Cys present in the peptide linker and the Cys at amino acid 84 of the class I HLA polypeptide.
50. The method of any one of aspects 45-49, wherein the peptide linker between the HPV16 cancer-associated peptide and the β2M polypeptide comprises the sequence CGGGS(GGGGS)n (SEQ ID NO: 68), GCGGS(GGGGS)n (SEQ ID NO:69), or GGCGS(GGGGS)n (SEQ ID NO:70), wherein n is an integer from 1-10.
51. The method of any one of aspects 30-50, wherein the β2M polypeptide comprises a Cys at amino acid 12, wherein the MHC class I heavy chain polypeptide comprises a Cys at amino acid 236, and wherein the TMP comprises a disulfide bond formed between the Cys at amino acid 12 of the β2M polypeptide and the Cys at amino acid 236 of the MHC class I heavy chain polypeptide.
52. The method of any one of aspects 8-29, wherein the TMP comprises two heterodimers, and wherein the heterodimers are joined by one or more disulfide bonds formed between the Ig Fc polypeptide of the first heterodimer and the Ig Fc polypeptide of the second heterodimer.
53. The method of aspect 52, wherein the two heterodimers have identical amino acid sequences.
54. The method of aspect 52 or 53, wherein the two heterodimers are joined by two disulfide bonds formed between the Ig Fc polypeptide of the first heterodimer and the Ig Fc polypeptide of the second heterodimer.
55. The method of any one of aspects 30-51, wherein the TMP comprises two single-chain polypeptides, and wherein the single-chain polypeptides are joined by one or more disulfide bonds formed between the Ig Fc polypeptide of the single-chain polypeptide and the Ig Fc polypeptide of the second single-chain polypeptide.
56. The method of aspect 55, wherein the two single-chain polypeptides have identical amino acid sequences.
57. The method of aspect 55 or 56, wherein the two single-chain polypeptides are joined by two disulfide bonds formed between the Ig Fc polypeptide of the first single-chain polypeptide and the Ig Fc polypeptide of the second single-chain polypeptide.
58. The method of any one of aspects 1-57, wherein the HPV16 cancer-associated peptide is an HPV16 cancer-associated peptide.
59. The method of aspect 58, wherein the HPV16 cancer-associated peptide is an HPV16 cancer-associated peptide is an HPV16 E6 epitope.
60. The method of aspect 59, wherein the cancer-associated peptide is selected from E6 18-26 (KLPQLCTEL; SEQ ID NO:24); E6 26-34 (LQTTIHDII; SEQ ID NO:25); E6 49-57 (VYDFAFRDL; SEQ ID NO:26); E6 52-60 (FAFRDLCIV; SEQ ID NO:27); E6 75-83 (KFYSKISEY; SEQ ID NO:28); and E6 80-88 (ISEYRHYCY; SEQ ID NO:29).
61. The method of aspect 58, wherein the cancer-associated peptide is an HPV16 cancer-associated peptide is an HPV16 E7 epitope.
62. The method of aspect 61, wherein the cancer-associated peptide is selected from E7 7-15 (TLHEYMLDL; SEQ ID NO:30); E7 11-19 (YMLDLQPET; SEQ ID NO:31); E7 11-20 (YMLDLQPETT; SEQ ID NO:20); E7 44-52 (QAEPDRAHY; SEQ ID NO:32); E7 49-57 (RAHYNIVTF (SEQ ID NO:33); E7 61-69 (CDSTLRLCV; SEQ ID NO:34); and E7 67-76 (LCVQSTHVDI; SEQ ID NO:35); E7 82-90 (LLMGTLGIV; SEQ ID NO:36); E7 86-93 (TLGIVCPI; SEQ ID NO:37); and E7 92-93 (LLMGTLGIVCPI; SEQ ID NO:38).
63. The method of aspect 62, wherein the cancer-associated peptide wherein the cancer-associated peptide is E7 11-20 (YMLDLQPETT; SEQ ID NO:20).
64. The method of any one of aspects 1-63, wherein the TMP is administered in an amount of from 0.5 mg/kg of body weight to 1 mg/kg of body weight.
65. The method of any one of aspects 1-63, wherein the TMP is administered in an amount of from 1 mg/kg of body weight to 5 mg/kg of body weight.
66. The method of any one of aspects 1-63, wherein the TMP is administered in an amount of from 5 mg/kg of body weight to 10 mg/kg of body weight.
67. The method of any one of aspects 1-63, wherein the TMP is administered in an amount of from 200 mg to 600 mg.
68. The method of any one of aspects 1-63, wherein the TMP is administered in an amount of from 250 mg to 450 mg.
69. The method of any one of aspects 1-63, wherein the TMP is administered in an amount of from 250 mg to 350 mg.
70. The method of any one of aspects 1-63, wherein the TMP is administered in an amount of 250 mg.
71. The method of any one of aspects 1-63, wherein the TMP is administered in an amount of 300 mg.
72. The method of any one of aspects 1-63, wherein the TMP is administered in an amount of 350 mg.
73. The method of any one of aspects 1-63, wherein the TMP is administered in an amount of 400 mg.
74. The method of any one of aspects 1-73, wherein the TMP is administered once every three weeks.
75. The method of any one of aspects 1-73, wherein the TMP is administered once every six weeks.
76. The method any one of aspects 1-75, wherein the PD-(L)1 CPI is an antibody that binds PD-1.
77. The method of aspect 76, wherein the PD-(L)1 CPI is pembrolizumab, nivolumab, cemiplimab, dostarlimab, retifanlimab, toripalimab or tislelizumab.
78. The method of aspect 77, wherein the PD-(L)1 CPI is pembrolizumab and is administered in an amount of 200 mg every three weeks or 400 mg every six weeks.
79. The method of aspect 76 or 77, wherein the PD-(L)1 CPI is nivolumab and is administered in an amount of 240 mg every 2 weeks or 480 mg every 4 weeks.
80. The method of any one of aspects 76-78, wherein the PD-(L)1 CPI is administered subcutaneously, optionally wherein the PD-(L)1 CPI is pembrolizumab or nivolumab.
81. The method of aspect 77, wherein the PD-(L)1 CPI is cemiplimab.
82. The method of aspect 77, wherein the PD-(L)1 CPI is dostarlimab.
83. The method of aspect 77, wherein the PD-(L)1 CPI is retifanlimab.
84. The method of aspect 77, wherein the PD-(L)1 CPI is toripalimab.
85. The method of aspect 77, wherein the PD-(L)1 CPI is tislelizumab.
86. The method of any one of aspects 1-75, wherein the PD-(L)1 CPI is an antibody that binds PD-L1.
87. The method of aspect 86, wherein the PD-(L)1 CPI is atezolizumab.
88. The method of aspect 86, wherein the PD-(L)1 CPI is avelumab.
89. The method of aspect 86, wherein the PD-(L)1 CPI is durvalumab
90. The method of any one of aspects 1-89, wherein the cancer is a newly diagnosed HPV16+ head and neck cancer, or is a recurrent and/or metastatic HPV16+ head and neck cancer.
91. The method of aspect 90, wherein the HPV16+ head and neck cancer is an oropharynx squamous cell cancer.
92. The method of aspect 90, wherein the HPV16+ head and neck cancer is an oropharyngeal cancer, nasopharyngeal cancer, hypopharyngeal cancer, and laryngeal cancer
93. The method of any of aspects 1-89, wherein the HPV16+ cancer is penile cancer.
94. The method of any of aspects 1-89, wherein the HPV16+ cancer is anal cancer.
95. The method of any of aspects 1-89, wherein the HPV16+ cancer is vaginal cancer.
96. The method of any of aspects 1-89, wherein the HPV16+ cancer is vulvar cancer.
97. The method of any of aspects 1-89, wherein the HPV16+ cancer is cervical cancer.

### EXAMPLE

The following example is put forth so as to provide those of ordinary skill in the art with a complete disclosure and description of how to make and use the methods of this disclosure, and is not intended to limit the scope of what the inventors regard as their invention set forth in the appended claims.

### Example 1

A Phase I dose escalation study of a TMP (CUE-101) in combination with pembrolizumab, designated KEYNOTE-A78, was conducted in patients with HPV16+ recurrent/metastatic head and neck squamous cell carcinoma (R/M HNSCC). CUE-101 is a homodimer of two heterodimers. Each heterodimer includes:
a) a first polypeptide comprising (i) the HPV16 E7 (11-20) peptide YMLDLQPETT, (ii) a linker comprising amino acid sequence GGGGSGGGGSGGGGS (SEQ ID NO:75), and (iii) a β2M polypeptide comprising an amino acid sequence as set forth in SEQ ID NO:2 (depicted in FIG. 7B; and
b) a second polypeptide comprising the amino acid sequence as set forth in SEQ ID NO: 17 (and depicted in FIG. 7Q), wherein the second polypeptide comprises (i) two copies of a variant IL-2 polypeptide, (ii) an MHC class I HLA-A*0201 heavy chain polypeptide, (iii) an Ig Fc polypeptide comprising LALA substitutions at the L14 and L15 amino acids (as discussed above), and peptide linkers interposed between the components of the second polypeptide. The Ig Fc optionally may exclude a C-terminal Lys; for example, the second polypeptide can comprise the amino acid sequence set forth in SEQ ID NO:18 (and depicted in FIG. 7R). The first and second polypeptides of CUE-101 are covalently linked to one another via a disulfide bond between a Cys residue at amino acid 12 of the β2M polypeptide and a Cys residue at amino acid 236 of the MHC class I heavy chain polypeptide. The two heterodimers are joined to each other by two disulfide bonds that join the Ig Fc polypeptide of one heterodimer to the Ig Fc polypeptide of the other heterodimer.

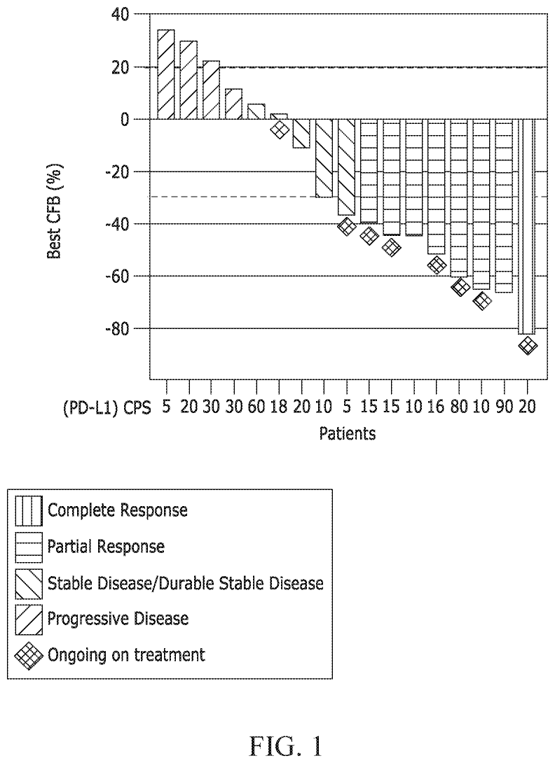

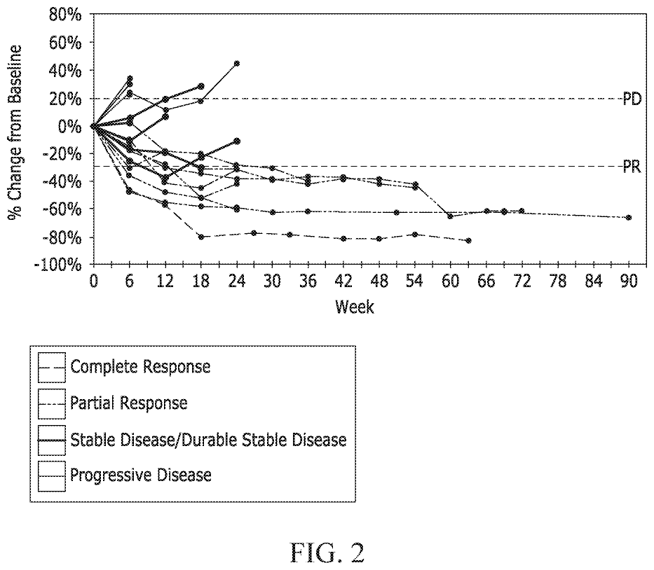

In the KEYNOTE-A78 study, to date twenty-two patients with newly diagnosed HPV16+ recurrent/metastatic head and neck squamous cell carcinoma received a combination therapy of 4 mg/kg CUE-101 and 200 mg pembrolizumab once every three weeks. Each of the combination-therapy patients had a PD-L1 CPS≥1. Data from the combination study for the first seventeen evaluable patients are shown in FIG. 1 and FIG. 2. The overall survival for the 22 patients as of the data cut-off is illustrated in FIG. 3.

FIG. 1 illustrates the best change from baseline in the sum of diameters of target lesions with best overall response as measured by RECIST 1.1. The Overall Response Rate (ORR) was 47%; the Disease Control Rate (DCR) was 65%.

FIG. 2 is a spider plot that shows change from baseline in the sum of diameters of target lesions as measured by RECIST 1.1.

FIG. 3 is a swimmer plot illustrating the overall survival (months from first dose) in patients treated with 4 mg/kg CUE-101 and pembrolizumab for 22 patients. Durable stable disease (SD) requires RECIST 1.1 SC on two or more consecutive scans. Response duration is indicated on the plot. At the time of the data cut-off, the Kaplan-Meier estimate of median PFS is 5.8 months [95% CI; 2.6, NA].

The data show that an Objective response rate of 47% (1 CR, 7 PR) was observed in newly diagnosed R/M patients treated with CUE-101 in combination with pembrolizumab. See FIG. 4. This ORR represents approximately a 240 percent increase compared to the reported ORR of 19% for patients with a PD-L1 CPS≥1 who were treated with pembrolizumab alone in the KEYNOTE-48 trial. See, Harrington et al., J. Clin. Oncol. 41:790-802 (2022), and Burtness et al. J. Clin. Oncol. 40:2321-2332 (2022). Complete Response and 5 out of 7 Partial Response (PRs) occurring in tumors with CPS of 20 or less and a DCR of 65%. Twenty-one of the 22 patients who received the combination of CUE-101 and pembrolizumab remain alive at time of data cut-off, including 8 patients for ≥12 months.

As shown in FIG. 5, as of the data cut-off date, “CPS-Low” patients, i.e., patients with a PD-L1 CPS between 1 and 19 treated with the combination of CUE-101 and pembrolizumab demonstrated an ORR of 56%. This ORR represents approximately a 380 percent increase compared to the reported ORR of 14.5% for patients with a PD-L1 CPS between 1 and 19 who were treated pembrolizumab alone in the KEYNOTE-48 trial. This result is particularly significant since patients with low CPS scores (CPS 1-19) represent approximately 50% of all patients that have a PD-L1 CPS≥1.

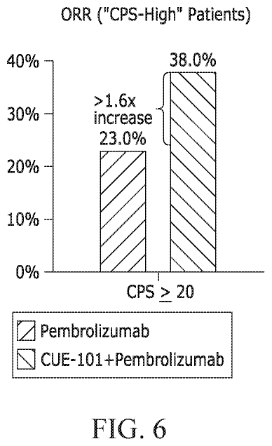

As shown in FIG. 6, as of the data cut-off date, “CPS-High” patients, i.e., patients with a PD-L1 CPS≥20 treated with the combination of CUE-101 and pembrolizumab demonstrated an ORR of 38%. This ORR represents approximately a 160 percent increase compared to the reported ORR of 23% for patients with a PD-L1 CPS≥20 who were treated pembrolizumab alone in the KEYNOTE-48 trial.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the claims appended hereto.

What is claimed is:

1. A method of treating an HPV16-associated cancer in an individual, the method comprising administering to the individual a combination of (i) a PD-(L)1 CPI, and (ii) a T cell modulating protein (TMP), wherein the TMP comprises:
i) an HPV16 cancer-associated peptide, wherein the HPV16 cancer-associated peptide is a peptide having a length of from 8 amino acids to 14 amino acids;
ii) a β2-microglobulin (β2M) polypeptide;
iii) one or more immunomodulatory polypeptides;
iv) a class I major histocompatibility complex (MHC) heavy chain polypeptide; and
v) a scaffold component, optionally wherein the scaffold component is an immunoglobulin (Ig) Fc polypeptide,
wherein the TMP may comprise one or more independently selected peptide linkers between any two of the components, and wherein when the TMP comprises more than one immunomodulatory polypeptide, the immunomodulatory polypeptides may be connected by one or more independently selected peptide linkers,
wherein the individual was previously determined to have progressive disease based on RECIST 1.1 criteria following (i) monotherapy with a PD-(L)1 CPI, or (ii) combination therapy comprising treatment with a PD-(L)1 CPI and one or more therapeutic agents (optionally one or more chemotherapeutic agents) other than the TMP, and
wherein the PD-(L)1 CPI and the TMP are administered at the same time or at different times.

2. The method of claim 1, wherein the cancer is associated with a PD-L1 Combined Positive Score (PD-L1 CPS) that is 20 or less, or 19 or less.

3. The method of claim 1, wherein the cancer is associated with a PD-L1 CPS that is 20 or greater, or 21 or greater.

4. The method of claim 1, wherein the cancer is associated with a PD-L1 CPS that is either: i) 0 (zero); or ii) more than 0 but less than 1.

5. A method of treating an HPV16-associated cancer in an individual, the method comprising administering to the individual a combination of (i) a PD-(L)1 CPI, and (ii) a T cell modulating protein (TMP), wherein the TMP comprises:
i) an HPV16 cancer-associated peptide, wherein the HPV16 cancer-associated peptide is a peptide having a length of from 8 amino acids to 14 amino acids;
ii) a β2-microglobulin (β2M) polypeptide;
iii) one or more immunomodulatory polypeptides;
iv) a class I major histocompatibility complex (MHC) heavy chain polypeptide; and
v) a scaffold component, optionally wherein the scaffold component is an immunoglobulin (Ig) Fc polypeptide,
wherein the TMP comprises one or more independently selected peptide linkers between any two of the components, and wherein when the TMP comprises more than one immunomodulatory polypeptide, the immunomodulatory polypeptides may be connected by one or more independently selected peptide linkers,
wherein the PD-(L)1 CPI and the protein are administered at the same time or at different times, and
wherein the cancer is associated with a PD-L1 Combined Positive Score (PD-L1 CPS) that is either 0 or more than 0 but less than 1.

6. The method of claim 5, wherein
the combination is administered to an individual who (i) had no prior therapies following an initial diagnosis of HPV16+ cancer, (ii) had at least one prior therapy following an initial diagnosis of HPV16+ cancer and was determined to have progressive disease, (iii) is receiving the combination as a neoadjuvant therapy for an HPV16+ cancer, (iv) is receiving the combination as a adjuvant therapy for an HPV16+ cancer, (v) is receiving the combination as a first line treatment following a diagnosis of recurrent and/or metastatic HPV16+ cancer, or (vi) is receiving the combination as a second line or beyond therapy following a diagnosis of recurrent and/or metastatic HPV16+ cancer and the patient has been diagnosed with progressive disease, and
wherein optionally the individual previously was not qualified for treatment with a PD-(L)1 CPI, either alone or in combination with one or more therapeutic agents, e.g., one or more chemotherapeutic agents.

7. The method of any one of claims 1-5, wherein the PD-L1 CPS is determined by an Approved Laboratory using:
(i) a PD-L1 IHC 22C3 pharmDx assay or an assay that employs a monoclonal mouse anti-PD-L1 antibody, clone 22C3;
(ii) a PD-L1 IHC 28-8 pharmDx assay or an assay that employs a monoclonal mouse anti-PD-L1 antibody, clone 28-8; or
(iii) an assay other than (i) or (ii).

8. The method of any one of claims 1-7, wherein the TMP comprises a heterodimer, wherein the heterodimer comprises:
a) a first polypeptide comprising:
i) the HPV16 cancer-associated peptide; and
ii) the β2-microglobulin (β2M) polypeptide; and
b) a second polypeptide comprising:
i) the one or more immunomodulatory polypeptides;
ii) the MHC class I heavy chain polypeptide; and
iii) an immunoglobulin (Ig) Fc polypeptide.

9. The method of claim 8, wherein:
a1) the first polypeptide comprises, in order from N-terminus to C-terminus:
i) the HPV16 cancer-associated peptide;
ii) a peptide linker; and
iii) the β2M polypeptide; and
b1) the second polypeptide comprises, in order from N-terminus to C-terminus:
i) the one or more immunomodulatory polypeptides;
ii) an optional linker;
iii) the MHC class I heavy chain polypeptide;
iv) an optional linker; and
v) the Ig Fc polypeptide, or
a2) the first polypeptide comprises, in order from N-terminus to C-terminus:
i) the HPV16 cancer-associated peptide;
ii) a peptide linker; and
iii) the β2M polypeptide; and
b2) the second polypeptide comprises, in order from N-terminus to C-terminus:
i) the MHC class I heavy chain polypeptide;
ii) an optional linker;
iii) the Ig Fc polypeptide;
iv) an optional linker; and
v) the one or more immunomodulatory polypeptides,
wherein when the TMP comprises more than one immunomodulatory polypeptide, the TMP may comprise one or more linkers between the immunomodulatory polypeptides.

10. The method of claim 8 or 9, wherein the heterodimer comprises a disulfide bond formed between a Cys residue in the β2M polypeptide and a Cys residue in the MHC class I heavy chain polypeptide.

11. The method of any one of claims 8-10, wherein the heterodimer comprises a disulfide bond formed between (i) a Cys residue in a Cys-containing linker interposed between the HPV16 cancer-associated peptide and the β2M polypeptide, and (ii) a Cys residue in the MHC class I heavy chain polypeptide.

12. The method of claim 8 or 9, wherein the first polypeptide and the second polypeptide are covalently linked to one another by at least a first and second disulfide bond,
wherein the first disulfide bond formed between (i) a Cys residue in a Cys-containing linker between the HPV16 cancer-associated peptide and the β2M polypeptide, and (ii) a Cys residue in the MHC class I heavy chain polypeptide; and
wherein the second disulfide bond is formed between a Cys residue in the β2M polypeptide and a Cys residue in the MHC class I heavy chain polypeptide.

13. The method of any one of claims 8-12, wherein the one or more immunomodulatory polypeptides are selected from the group of a cytokine, a 4-1BBL polypeptide, an ICOS-L polypeptide, an OX-40L polypeptide, a CD80 polypeptide, a CD86 polypeptide, a CD40 polypeptide, a CD70 polypeptide, and combinations thereof.

14. The method of any one of claims 8-13, wherein at least one of the one or more immunomodulatory polypeptides is an IL-2 polypeptide.

15. The method of claim 14, wherein at least one of the one or more immunomodulatory polypeptides comprise a variant IL-2 polypeptide that exhibits reduced affinity to an IL-2 receptor compared to the affinity of a wild-type IL-2 polypeptide for the IL-2 receptor.

16. The method of claim 15, wherein the variant IL-2 polypeptide has at least 95% amino acid sequence identity to the amino acid sequence set forth in SEQ ID NO:21, wherein amino acid 16 is other than histidine and amino acid 42 is other than phenylalanine.

17. The method of claim 15, wherein the variant IL-2 polypeptide has at least 98% amino acid sequence identity to the amino acid sequence set forth in SEQ ID NO:21, wherein amino acid 16 is other than histidine and amino acid 42 is other than phenylalanine.

18. The method of claim 16 or 17, wherein amino acid 16 is Ala, Thr, Asp or Glu.

19. The method of any one of claims 16-18, wherein amino acid 42 is Ala.

20. The method of claim 16 or 17, wherein amino acid 16 is Ala and amino acid 42 is Ala.

21. The method of any one of claims 1-20, wherein the TMP comprises a variant Ig Fc polypeptide comprising one or more amino acid substitutions that reduce or substantially eliminate antibody-dependent cell-mediated cytotoxicity (ADCC) and/or complement-dependent cytotoxicity (CDC).

22. The method of any one of claims 1-21, wherein the TMP comprises a variant Ig Fc polypeptide comprising an amino acid sequence having at least about 95% amino acid sequence identity to the amino acid sequence set forth in any one of SEQ ID NOs: 7-10 (FIG. 7G-7J).

23. The method of claim 22, wherein the TMP comprises a variant Ig Fc polypeptide comprising an amino acid sequence having at least about 98% amino acid sequence identity to the amino acid sequence set forth in SEQ ID NO:9 or SEQ ID NO: 10, and comprises an Ala at amino acid 14 and an Ala at amino acid 15, based on the amino acid numbering depicted in SEQ ID NO:9.

24. The method of any one of claims 1-23, wherein the TMP comprises at least two immunomodulatory polypeptides, and wherein at least two of the immunomodulatory polypeptides are the same.

25. The method of any of claims 15-23, wherein the TMP comprises two variant IL-2 polypeptides in tandem, and wherein the two variant IL-2 polypeptides are optionally connected by a linker.

26. The method of claim 25, wherein
a) the first polypeptide of each heterodimer comprises, in order from N-terminus to C-terminus:
i) the HPV16 cancer-associated peptide, wherein the peptide has a length of 8-12 amino acids;
ii) a peptide linker; and
iii) the β2M polypeptide, wherein the β2M polypeptide comprises an amino acid sequence having at least 95% amino acid sequence identity to the amino acid sequence set forth in SEQ ID NO:1 or SEQ ID NO:2;
b) the second polypeptide of each heterodimer comprises, in order from N-terminus to C-terminus:
i) the first variant IL-2 polypeptide;
ii) a second variant IL-2 polypeptide;
iii) the MHC class I heavy chain polypeptide, wherein the MHC class I heavy chain polypeptide comprises an amino acid sequence having at least 95% amino acid sequence identity to the amino acid sequence set forth in SEQ ID NO:3; and
iv) the Ig Fc polypeptide,
wherein each variant IL-2 polypeptide has at least 95% amino acid sequence identity to the amino acid sequence set forth in SEQ ID NO:21, wherein amino acid 16 is other than histidine and amino acid 42 is other than phenylalanine, and
wherein the second polypeptide comprises an independently selected peptide linker between one or more of:
a) the first immunomodulatory polypeptide and the second immunomodulatory polypeptide;
b) the second immunomodulatory polypeptide and the MHC class I heavy chain polypeptide; and
c) the MHC class I heavy chain polypeptide and the Ig Fc polypeptide.

27. The method of claim 25, wherein
a) the first polypeptide of each heterodimer comprises, in order from N-terminus to C-terminus:
i) the HPV16 cancer-associated peptide, wherein the peptide has a length of 8-12 amino acids;
ii) a peptide linker; and
iii) the β2M polypeptide, wherein the β2M polypeptide comprises an amino acid sequence having at least 95% amino acid sequence identity to the amino acid sequence set forth in SEQ ID NO:1 or SEQ ID NO:2;
b) the second polypeptide of each heterodimer comprises, in order from N-terminus to C-terminus:
i) the MHC class I heavy chain polypeptide, wherein the MHC class I heavy chain polypeptide comprises an amino acid sequence having at least 95% amino acid sequence identity to the amino acid sequence set forth in SEQ ID NO:3;
ii) the Ig Fc polypeptide;
iii) the first variant IL-2 polypeptide; and
iv) a second variant IL-2 polypeptide,
wherein each variant IL-2 polypeptide has at least 95% amino acid sequence identity to the amino acid sequence set forth in SEQ ID NO:21, wherein amino acid 16 is other than histidine and amino acid 42 is other than phenylalanine, and
wherein the second polypeptide comprises an independently selected peptide linker between one or more of:
a) the MHC class I heavy chain polypeptide and the Ig Fc polypeptide
b) the Ig Fc polypeptide the first immunomodulatory polypeptide; and
c) the first immunomodulatory polypeptide and the second immunomodulatory polypeptide.

28. The method of claim 26 or 27, wherein:
i) the β2M polypeptide comprises a Cys at amino acid 12 based on the amino acid numbering of SEQ ID NO:2; and
ii) the class I MHC polypeptide comprises a Cys at residue 236 based on the amino acid numbering of SEQ ID NO:4,
wherein each heterodimer comprises a disulfide bond linking the first polypeptide to the second polypeptide, and wherein the disulfide bond joins the Cys at amino acid 12 of the β2M polypeptide to the Cys at amino acid 236 of the MHC heavy chain polypeptide, and
wherein the Ig Fc polypeptide comprises an amino acid sequence having at least about 95% amino acid sequence identity to the amino acid sequence set forth in SEQ ID NO:9 or SEQ ID NO:10.

29. The method of claim 28, wherein:
i) the β2M polypeptide comprises the amino acid sequence set forth in SEQ ID NO:2;
ii) the first and second variant IL-2 polypeptides comprise the amino acid sequence set forth in SEQ ID NO:22;
iii) the MHC class I heavy chain polypeptide comprises the amino acid sequence set forth in SEQ ID NO:4; and
iv) the Ig Fc polypeptide comprises an amino acid sequence having at least about 98% amino acid sequence identity to the amino acid sequence set forth in SEQ ID NO:9 or SEQ ID NO: 10, and comprises an Ala at amino acid 14 and an Ala at amino acid 15 based on the amino acid numbering depicted in SEQ ID NO:9, and
wherein the second polypeptide comprises a peptide linker between:
a) the first and second immunomodulatory polypeptides;
b) the second variant immunomodulatory polypeptide and the MHC class I heavy chain polypeptide; and
c) the MHC class I heavy chain polypeptide and the Ig Fc polypeptide.

30. The method of any one of claims 1-7, wherein the TMP comprises a single-chain polypeptide, and wherein the single-chain polypeptide comprises from N-terminus to C-terminus:
i) the HPV16 cancer-associated peptide;
ii) an optional peptide linker;
iii) the β2M polypeptide;
iv) an optional peptide linker;
v) the MHC class I heavy chain polypeptide;
vi) an optional peptide linker;
vii) an immunoglobulin (Ig) Fc polypeptide;
viii) an optional peptide linker; and
ix) the one or more immunomodulatory polypeptides.

31. The method of claim 30, wherein the one or more immunomodulatory polypeptides are selected from the group of a cytokine, a 4-1BBL polypeptide, an ICOS-L polypeptide, an OX-40L polypeptide, a CD80 polypeptide, a CD86 polypeptide, a CD40 polypeptide, a CD70 polypeptide, and combinations thereof.

32. The method of claim 31, wherein at least one of the one or more immunomodulatory polypeptides is an IL-2 polypeptide.

33. The method of claim 32, wherein at least one of the one or more immunomodulatory polypeptides comprise a variant IL-2 polypeptide that exhibits reduced affinity to an IL-2 receptor compared to the affinity of a wild-type IL-2 polypeptide for the IL-2 receptor.

34. The method of claim 33, wherein the variant IL-2 polypeptide variant IL-2 polypeptide has at least 95% amino acid sequence identity to the amino acid sequence set forth in SEQ ID NO:21, wherein amino acid 16 is other than histidine and amino acid 42 is other than phenylalanine.

35. The method of claim 34, wherein the variant IL-2 polypeptide has at least 98% amino acid sequence identity to the amino acid sequence set forth in SEQ ID NO:21, wherein amino acid 16 is other than histidine and amino acid 42 is other than phenylalanine.

36. The method of claim 34 or 35, wherein amino acid 16 is Ala, Thr, Asp or Glu.

37. The method of any one of claims 34-36, wherein amino acid 42 is Ala.

38. The method of claim 36 or 37, wherein amino acid 16 is Ala and amino acid 42 is Ala.

39. The method of any one of claims 30-38, wherein the TMP comprises at least two immunomodulatory polypeptides, and wherein at least two of the immunomodulatory polypeptides are the same.

40. The method of any of claims 33-38, wherein the TMP comprises two variant IL-2 polypeptides in tandem, and wherein the two variant IL-2 polypeptides are optionally connected by a linker.

41. The method of any one of claims 30-40, wherein
the β2M polypeptide comprises an amino acid sequence having at least 95% amino acid sequence identity to the amino acid sequence set forth in SEQ ID NO: 1 or SEQ ID NO:2, and the MHC class I heavy chain polypeptide comprises an amino acid sequence having at least 95% amino acid sequence identity to the amino acid sequence set forth in SEQ ID NO:3.

42. The method of any one of claims 30-41, wherein the Ig Fc polypeptide comprises an amino acid sequence having at least about 95% amino acid sequence identity to the amino acid sequence set forth in any one of SEQ ID NOs: 7-10.

43. The method of claim 42, wherein the Ig Fc polypeptide is a variant Ig Fc polypeptide comprises one or more amino acid substitutions that reduce or substantially eliminate antibody-dependent cell-mediated cytotoxicity (ADCC) and/or complement-dependent cytotoxicity (CDC).

44. The method of claim 43, wherein the Ig Fc polypeptide comprises an amino acid sequence having at least about 98% amino acid sequence identity to the amino acid sequence set forth in SEQ ID NO:9 or SEQ ID NO: 10, and comprises an Ala at amino acid 14 and an Ala at amino acid 15 based on the amino acid numbering depicted in SEQ ID NO:9.

45. The method of any one of claims 30-44, wherein the TMP comprises a Cys-containing peptide linker between the cancer-associated peptide and the β2M polypeptide, and wherein the TMP comprises an intrachain disulfide bond between the Cys present in the peptide linker and a Cys in the MHC class I heavy chain polypeptide.

46. The method of claim 45, wherein the MHC class I heavy chain polypeptide comprises a Cys at any one of amino acids 135-143, based on the numbering of the MHC class I heavy chain polypeptide set forth in SEQ ID NO:6 (depicted in FIG. 7F), and wherein amino acid 84, based on the numbering of the MHC class I heavy chain polypeptide depicted in FIG. 7F, is other than Cys; and wherein the TMP comprises an intrachain disulfide bond between the Cys present in the peptide linker and the Cys at any one of amino acids 135-143 of the MHC class I heavy chain polypeptide.

47. The method of claim 45, wherein the MHC class I heavy chain polypeptide comprises a Cys at amino acid 138, 139, or 140 based on the numbering of the MHC class I heavy chain polypeptide set forth in SEQ ID NO:6 (depicted in FIG. 7F).

48. The method of claim 45, wherein the MHC class I heavy chain polypeptide comprises a Cys at amino acid 139 based on the numbering of the MHC class I heavy chain polypeptide set forth in SEQ ID NO:6 (depicted in FIG. 7F).

49. The method of claim 45, wherein the TMP comprises an intrachain disulfide bond between the Cys present in the peptide linker and the Cys at amino acid 84 of the class I HLA polypeptide.

50. The method of any one of claims 45-49, wherein the peptide linker between the HPV16 cancer-associated peptide and the β2M polypeptide comprises the sequence CGGGS(GGGGS)n (SEQ ID NO: 68), GCGGS(GGGGS)n (SEQ ID NO:69), or GGCGS(GGGGS)n (SEQ ID NO:70), wherein n is an integer from 1-10.

51. The method of any one of claims 30-50, wherein the β2M polypeptide comprises a Cys at amino acid 12, wherein the MHC class I heavy chain polypeptide comprises a Cys at amino acid 236, and wherein the TMP comprises a disulfide bond formed between the Cys at amino acid 12 of the β2M polypeptide and the Cys at amino acid 236 of the MHC class I heavy chain polypeptide.

52. The method of any one of claims 8-29, wherein the TMP comprises two heterodimers, and wherein the heterodimers are joined by one or more disulfide bonds formed between the Ig Fc polypeptide of the first heterodimer and the Ig Fc polypeptide of the second heterodimer.

53. The method of claim 52, wherein the two heterodimers have identical amino acid sequences.

54. The method of claim 52 or 53, wherein the two heterodimers are joined by two disulfide bonds formed between the Ig Fc polypeptide of the first heterodimer and the Ig Fc polypeptide of the second heterodimer.

55. The method of any one of claims 30-51, wherein the TMP comprises two single-chain polypeptides, and wherein the single-chain polypeptides are joined by one or more disulfide bonds formed between the Ig Fc polypeptide of the single-chain polypeptide and the Ig Fc polypeptide of the second single-chain polypeptide.

56. The method of claim 55, wherein the two single-chain polypeptides have identical amino acid sequences.

57. The method of claim 55 or 56, wherein the two single-chain polypeptides are joined by two disulfide bonds formed between the Ig Fc polypeptide of the first single-chain polypeptide and the Ig Fc polypeptide of the second single-chain polypeptide.

58. The method of any one of claims 1-57, wherein the HPV16 cancer-associated peptide is an HPV16 cancer-associated peptide.

59. The method of claim 58, wherein the HPV16 cancer-associated peptide is an HPV16 cancer-associated peptide is an HPV16 E6 epitope.

60. The method of claim 59, wherein the cancer-associated peptide is selected from E6 18-26 (KLPQLCTEL; SEQ ID NO:24); E6 26-34 (LQTTIHDII; SEQ ID NO:25); E6 49-57 (VYDFAFRDL; SEQ ID NO:26); E6 52-60 (FAFRDLCIV; SEQ ID NO:27); E6 75-83 (KFYSKISEY; SEQ ID NO:28); and E6 80-88 (ISEYRHYCY; SEQ ID NO:29).

61. The method of claim 58, wherein the cancer-associated peptide is an HPV16 cancer-associated peptide is an HPV16 E7 epitope.

62. The method of claim 61, wherein the cancer-associated peptide is selected from E7 7-15 (TLHEYMLDL; SEQ ID NO:30); E7 11-19 (YMLDLQPET; SEQ ID NO:31); E7 11-20 (YMLDLQPETT; SEQ ID NO:20); E7 44-52 (QAEPDRAHY; SEQ ID NO:32); E7 49-57 (RAHYNIVTF (SEQ ID NO:33); E7 61-69 (CDSTLRLCV; SEQ ID NO:34); and E7 67-76 (LCVQSTHVDI; SEQ ID NO:35); E7 82-90 (LLMGTLGIV; SEQ ID NO:36); E7 86-93 (TLGIVCPI; SEQ ID NO:37); and E7 92-93 (LLMGTLGIVCPI; SEQ ID NO:38).

63. The method of claim 62, wherein the cancer-associated peptide wherein the cancer-associated peptide is E7 11-20 (YMLDLQPETT; SEQ ID NO:20).

64. The method of any one of claims 1-63, wherein the TMP is administered in an amount of from 0.5 mg/kg of body weight to 1 mg/kg of body weight.

65. The method of any one of claims 1-63, wherein the TMP is administered in an amount of from 1 mg/kg of body weight to 5 mg/kg of body weight.

66. The method of any one of claims 1-63, wherein the TMP is administered in an amount of from 5 mg/kg of body weight to 10 mg/kg of body weight.

67. The method of any one of claims 1-63, wherein the TMP is administered in an amount of from 200 mg to 600 mg.

68. The method of any one of claims 1-63, wherein the TMP is administered in an amount of from 250 mg to 450 mg.

69. The method of any one of claims 1-63, wherein the TMP is administered in an amount of from 250 mg to 350 mg.

70. The method of any one of claims 1-63, wherein the TMP is administered in an amount of 250 mg.

71. The method of any one of claims 1-63, wherein the TMP is administered in an amount of 300 mg.

72. The method of any one of claims 1-63, wherein the TMP is administered in an amount of 350 mg.

73. The method of any one of claims 1-63, wherein the TMP is administered in an amount of 400 mg.

74. The method of any one of claims 1-73, wherein the TMP is administered once every three weeks.

75. The method of any one of claims 1-73, wherein the TMP is administered once every six weeks.

76. The method any one of claims 1-75, wherein the PD-(L)1 CPI is an antibody that binds PD-1.

77. The method of claim 76, wherein the PD-(L)1 CPI is pembrolizumab, nivolumab, cemiplimab, dostarlimab, retifanlimab, toripalimab or tislelizumab.

78. The method of claim 77, wherein the PD-(L)1 CPI is pembrolizumab and is administered in an amount of 200 mg every three weeks or 400 mg every six weeks.

79. The method of claim 76 or 77, wherein the PD-(L)1 CPI is nivolumab and is administered in an amount of 240 mg every 2 weeks or 480 mg every 4 weeks.

80. The method of any one of claims 76-78, wherein the PD-(L)1 CPI is administered subcutaneously, optionally wherein the PD-(L)1 CPI is pembrolizumab or nivolumab.

81. The method of claim 77, wherein the PD-(L)1 CPI is cemiplimab.

82. The method of claim 77, wherein the PD-(L)1 CPI is dostarlimab.

83. The method of claim 77, wherein the PD-(L)1 CPI is retifanlimab.

84. The method of claim 77, wherein the PD-(L)1 CPI is toripalimab.

85. The method of claim 77, wherein the PD-(L)1 CPI is tislelizumab.

86. The method of any one of claims 1-75, wherein the PD-(L)1 CPI is an antibody that binds PD-L1.

87. The method of claim 86, wherein the PD-(L)1 CPI is atezolizumab.

88. The method of claim 86, wherein the PD-(L)1 CPI is avelumab.

89. The method of claim 86, wherein the PD-(L)1 CPI is durvalumab

90. The method of any one of claims 1-89, wherein the cancer is a newly diagnosed HPV16+ head and neck cancer, or is a recurrent and/or metastatic HPV16+ head and neck cancer.

91. The method of claim 90, wherein the HPV16+ head and neck cancer is an oropharynx squamous cell cancer.

92. The method of claim 90, wherein the HPV16+ head and neck cancer is an oropharyngeal cancer, nasopharyngeal cancer, hypopharyngeal cancer, and laryngeal cancer

93. The method of any of claims 1-89, wherein the HPV16+ cancer is penile cancer.

94. The method of any of claims 1-89, wherein the HPV16+ cancer is anal cancer.

95. The method of any of claims 1-89, wherein the HPV16+ cancer is vaginal cancer.

96. The method of any of claims 1-89, wherein the HPV16+ cancer is vulvar cancer.

97. The method of any of claims 1-89, wherein the HPV16+ cancer is cervical cancer.
